(19) United States
(12) Patent Application Publication
(10) Pub. No.: US 2005/0261711 A1
(43) Pub. Date: Nov. 24, 2005

(54) Treatment method and endoscope apparatus

(75) Inventors: Yuta Okada, Hachioji-shi (JP); Keita Suzuki, Kokubunji-shi (JP); Satoshi Miyamoto, Nishitama-gun (JP); Takayasu Mikkaichi, Fuchu-shi (JP); Raifu Matsui, Hino-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 10/852,759

(22) Filed: May 24, 2004

(51) Int. Cl. A61B 17/08

(52) U.S. Cl. 606/153

(57) ABSTRACT

According to a treatment method of the present invention, an anterior wall of stomach and a posterior wall of stomach are pulled to each other, and a part of an inner surface of the anterior wall and a part of an inner surface of the posterior wall are joined to each other to extend an esophagus. A false esophagus is thereby formed. A part of a peritoneal cavity side of a fundus of stomach is made to perorally approach a part of a peritoneal cavity side of the false esophagus. The part of the fundus of stomach and the part of the false esophagus are perorally fixed to each other.

### BACKGROUND OF THE INVENTION

1. Field of the Invention

This invention relates to a treatment method of a gastroesophageal reflux disease and an endoscope apparatus for use in the treatment method.

2. Description of the Related Art

Gastroesophageal reflux disease (hereinafter referred to as GERD) representing reflux of a gastric acid in stomach to an esophagus has been known. GERD is considered to be caused by hypofunction in prevention of a reflux in a cardiac region due to abnormality in a lower esophageal sphincter.

A surgical treatment method called Nissen operation has been known as the treatment method of the GERD. The Nissen operation reconstitutes the cardiac region by surgically winding a fundus of stomach round an esophagus inside a peritoneal cavity. The esophagus is suppressed and closed by expanding the fundus of stomach wound round the esophagus. Thus, the reflux is prevented.

In addition, a treatment method of U.S. Pat. No. 5,088,979 has been known as the other treatment of the GERD. This treatment method perorally pushes an esophagus into a stomach from the esophagus side, and fixes the esophagus on the stomach such that the pushed esophagus is kept inside the stomach. The pushed esophagus is suppressed by expanding of the stomach in the vicinity of a gastroesophageal boundary.

Moreover, a treatment method of U.S. Pat. No. 6,312,437 has been known as the other treatment of the GERD. This treatment method pulls down a gastroesophageal junction into a stomach, pulls a fundus of stomach toward an esophagus and fixes the fundus of stomach on the esophagus. At this time, tissues in the vicinity of the gastroesophageal boundary are joined on the pulled tissues. The pulled gastroesophageal junction is suppressed by expanding of the stomach in the vicinity of the gastroesophageal boundary.

### BRIEF SUMMARY OF THE INVENTION

According to a treatment method of the present invention, perorally, an anterior wall of stomach and a posterior wall of stomach are pulled to each other, and a part of an inner surface of the anterior wall of stomach and a part of an inner surface of the posterior wall of stomach are joined to each other to extend an esophagus. A false esophagus is thereby formed. After that, a part of a peritoneal cavity side of a fundus of stomach is made to perorally approach a part of a peritoneal cavity side of the false esophagus. Subsequently, the part of the fundus of stomach and the part of the false esophagus which are made to approach are perorally fixed to each other.

An endoscope apparatus of the present invention is perorally inserted into a stomach to form a false esophagus in the stomach by pulling an anterior wall of stomach and a posterior wall of stomach to each other and joining a part of an inner surface of the anterior wall and a part of an inner surface of the posterior wall to each other so as to extend an esophagus.

An endoscope apparatus of the present invention comprises an insertion portion perorally inserted into a stomach. An observing optical system for observation of a body cavity is provided at the insertion portion. A tube-like member is provided integrally or separately at a distal portion of the insertion portion. A communication portion is formed to an outer peripheral wall of the tube-like member to make the interior of the tube-like member communicate with an exterior thereof. A first channel and a second channel are formed at the insertion portion or provided at the insertion portion so as to open to an inner side of the tube-like member. The endoscope apparatus further comprises a retained member and a retaining forceps. The retained member includes a first engaging portion and a second engaging portion which are engaged on a body wall. The retaining forceps includes the retained member at a distal portion thereof, being inserted into the first channel or the second channel, projecting the retained member from the interior of the tube-like member to the exterior thereof through the communication portion, engaging one of the first and second engaging portions with one of the anterior wall and the posterior wall, engaging the other of the first and second engaging portions with the other of the anterior wall and the posterior wall, and retaining the retained member with the first or second engaging portions being engaged on the anterior wall or the posterior wall.

An endoscope apparatus of the present invention comprises an insertion portion perorally inserted into a stomach. An observing optical system for observation of a body cavity is provided at the insertion portion. A channel is formed at the insertion portion or provided at the insertion portion. The endoscope apparatus further comprises a retained member and a retaining forceps. The retained member includes a first engaging portion and a second engaging portion which are engaged with a body wall. The retaining forceps includes the retained member at a distal portion thereof. The retaining forceps, which is inserted into the channel, engages one of the first and second engaging portions with one of an anterior wall of stomach and the posterior wall of stomach, moving the retained member with one of the first and second engaging portions being engaged with one of the anterior wall and the posterior wall, engaging the other one of the first and second engaging portions with the other one of the anterior wall and the posterior wall, and retaining the retained member with the first and second engaging portions being engaged on the anterior wall and the posterior wall.

An endoscope apparatus of the present invention comprises an insertion portion perorally inserted into a stomach. An observing optical system for observation of a body cavity is provided at the insertion portion. A channel is formed at the insertion portion or provided at the insertion portion. The endoscope apparatus further comprises a retained member and a retaining forceps. The retained member includes a first engaging portion and a second engaging portion which are engaged on a body wall, and a connecting portion connecting the first engaging portion and the second engaging portion and adjusting a distance between the first engaging portion and the second engaging portion. The retaining forceps includes the retained member at a distal portion thereof. The retaining forceps, which is inserted into the channel, engages one of the first and second engaging portions with one of an anterior wall of stomach and the posterior wall of stomach, engaging the other one of the first and second engaging portions on the other one of the anterior wall and the posterior wall, operating the connecting portion to adjust the distance between the first engaging portion and the second engaging portion with the first and second engaging portions being engaged with the anterior wall and the posterior wall, and retaining the retained member with the first and second engaging portions being engaged with the anterior wall and the posterior wall and the distance between the first engaging portion and the second engaging portion being adjusted.

An endoscope apparatus of the present invention comprises an insertion portion perorally inserted into a stomach. An observing optical system for observation of a body cavity is provided at the insertion portion. A tube-like member is provided integrally or separately at a distal portion At least one first communication portion is formed to an outer peripheral wall of the tube-like member. The first communication portion makes the interior and exterior of the tube-like member communicate with each other. In addition, at least one second communication portion is formed to an outer peripheral wall of the tube-like member. The second communication portion makes the interior and exterior of the tube-like member communicate with each other. A first channel and a second channel are formed at the insertion portion or provided at the insertion portion so as to open to the inner side of the tube-like member. An anterior wall of stomach is pulled through the first communication portion and a posterior wall of stomach is pulled through the second communication portion, from the exterior of the tube-like member into the interior thereof, by suction through one of the first channel and the second channel. The endoscope apparatus further comprises a retained member and a retaining forceps. The retained member includes a first engaging portion and a second engaging portion which are engaged with a body wall, and connecting member connecting the first engaging portion and the second engaging portion. The retaining forceps includes the retained member at a distal portion thereof, being inserted into the other of the first channel and the second channel. The retaining forceps engages one of the first and second engaging portions with one of the anterior wall and the posterior wall pulled into the interior of the tube-like member, engages the other of the first and second engaging portions with the other of the anterior wall and the posterior wall pulled into the interior of the tube-like member, and retains the retained member with the first or second engaging portions being engaged with the anterior wall or the posterior wall.

An endoscope apparatus of the present invention is perorally inserted into a stomach. The endoscope apparatus makes a part of a peritoneal cavity side of a fundus of stomach approach a part of the peritoneal cavity side of a false esophagus formed in the stomach, by pulling an anterior wall of stomach and a posterior wall of stomach to each other and joining a part of an inner surface of the anterior wall of stomach and a part of an inner surface of the posterior wall of stomach to each other so as to extend an esophagus.

An endoscope apparatus of the present invention comprises an insertion portion perorally inserted into a stomach. A bending portion operated to bend is provided at the insertion portion. An engaging portion to be engaged with an inner surface of the fundus of stomach is provided on the distal portion of the insertion portion.

An endoscope apparatus of the present invention is perorally inserted into a stomach. The endoscope apparatus fixes a part of a peritoneal cavity side of a false esophagus formed in the stomach by pulling an anterior wall of stomach and a posterior wall of stomach to each other and joining a part of an inner surface of the anterior wall of stomach and a part of an inner surface of the posterior wall of stomach to each other so as to extend an esophagus and a part of a peritoneal cavity side of a fundus of stomach made to approach the part of the peritoneal cavity side of the false esophagus.

An endoscope apparatus of the present invention comprises an insertion portion perorally inserted into a stomach. An observing optical system for observation of a body cavity is provided at the insertion portion. A tube-like member is provided integrally or separately at a distal portion of the insertion portion. A communication portion is formed to an outer peripheral wall of the tube-like member to make an interior of the tube-like member communicate with an exterior thereof. A first channel and a second channel are formed at the insertion portion or provided at the insertion portion so as to open to an inner side of the tube-like member. The endoscope apparatus further comprises a pulling forceps, retained member, and a retaining forceps. The pulling forceps is inserted into one of the first channel and the second channel, and pulls a part of a body wall of the fundus of stomach and a part of a body wall of the false esophagus into the interior of the tube-like member through the communication portion with the part of the body wall of the fundus of stomach and the part of the body wall of the false esophagus being mutually stacked. The retained member can fix the part of the body wall of the fundus of stomach and the part of the body wall of the false esophagus with the part of the body wall of the fundus of stomach and the part of the body wall of the false esophagus being mutually stacked. The retained member is provided on a distal portion of the retaining forceps. The retaining forceps is inserted into the other of the first channel and the second channel, and fixes the part of the body wall of the fundus of stomach and the part of the body wall of the false esophagus pulled into the tube-like member, inside the tube-like member, by retaining the retained member.

An endoscope apparatus of the present invention is perorally inserted into a stomach. The endoscope apparatus winds a fundus of stomach around a peritoneal cavity side of a false esophagus formed in the stomach by pulling an anterior wall of stomach and a posterior wall of stomach to each other and joining a part of an inner surface of the anterior wall of stomach and a part of an inner surface of the posterior wall of stomach to each other so as to extend an esophagus.

An endoscope apparatus of the present invention comprises an insertion portion perorally inserted into a stomach. A bending portion operated to bend is provided at the insertion portion. An engaging portion to be engaged with an inner surface of the fundus of stomach is provided on the distal portion of the insertion portion. An operating portion to rotate the endoscope about a central axis of the endoscope is provided on the proximal portion of the endoscope.

An endoscope apparatus of the present invention comprises a tube-like member to be perorally inserted into a stomach. A first bending portion operated to bend is provided at the tube-like member. The endoscope apparatus further comprises an insertion portion which is inserted into the tube-like member so as to freely advance or retreat in the tube-like member and which can project from a distal portion of the tube-like member. A second bending portion operated to bend is provided at the insertion portion.

An endoscope apparatus of the present invention comprises means for perorally forming a false esophagus in the stomach by pulling an anterior wall of stomach and a posterior wall of stomach to each other and joining a part of an inner surface of the anterior wall of stomach and a part of an inner surface of the posterior wall of stomach to each other so as to extend an esophagus, means for perorally making a part of a peritoneal cavity side of a fundus of stomach approach a part of a peritoneal cavity side of a false esophagus, and means for perorally fixing the part of the fundus of stomach and the part of the false esophagus which are made to approach to each other.

An endoscope apparatus of the present invention comprises means for perorally forming a false esophagus in the stomach by pulling an anterior wall of stomach and a posterior wall of stomach to each other and joining a part of an inner surface of the anterior wall of stomach and a part of an inner surface of the posterior wall of stomach to each other so as to extend an esophagus, means for perorally winding a fundus of stomach around a peritoneal cavity side of the false esophagus, and means for perorally fixing the part of the wound fundus of stomach and the part of the false esophagus to each other.

An endoscope apparatus of the present invention comprises an insertion portion perorally inserted into a stomach. An observing optical system for observation of a body cavity is provided at the insertion portion. A tube-like member is provided integrally or separately at a distal portion of the insertion portion. A first channel and a second channel open to an inner side of the tube-like member. A communication portion is formed to an outer peripheral wall of the tube-like member to make the interior of the tube-like member communicate with an exterior thereof. The endoscope apparatus further comprises a first retained member and a retaining forceps. The first retained member includes first and second engaging portions engaged with a body wall. The retaining forceps includes the first retained member at a distal portion thereof. The retaining forceps is inserted into the first channel. The retaining forceps projects the first retained member from the interior of the tube-like member to the exterior thereof through the communication portion, engaging one of the first and second engaging portions with one of the anterior wall and the posterior wall, engaging the other of the first and second engaging portions with the other of the anterior wall and the posterior wall, and retaining the first retained member with the first and second engaging portions being engaged with the anterior wall and the posterior wall. A bending portion operated to bend is provided at the insertion portion. An engaging portion engaged with an inner surface of a fundus of stomach is provided on the tube-like member. The endoscope apparatus further comprises a pulling forceps, a second retained member, and a retaining forceps. The pulling forceps is inserted into the first channel. The pulling forceps pulls a part of a body wall of the fundus of stomach and a part of a body wall of a false esophagus into the interior of the tube-like member through the communication portion with the part of the body wall of the fundus of stomach and the part of the body wall of the false esophagus being mutually stacked. The second retained member is capable of fixing the part of the body wall of the fundus of stomach and the part of the body wall of the false esophagus with the part of the body wall of the fundus of stomach and the part of the body wall of the false esophagus being mutually stacked. The retaining forceps includes the second retained member at a distal portion thereof. The retaining forceps is inserted into the second channel. The retaining forceps fixes the part of the body wall of the fundus of stomach and the part of the body wall of the false esophagus pulled into the tube-like member, inside the tube-like member, by retaining the second retained member.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is an anatomic illustration of a periphery of an esophagus and a stomach;

FIG. 2A is an illustration showing formation of a false esophagus in a treatment method according to a first embodiment of the present invention;

FIG. 2B is a cross-sectional view showing the false esophagus and the stomach as seen along a line IIB-IIB of FIG. 2A;

FIG. 2C is an illustration showing winding a fundus of stomach round the false esophagus from a peritoneal cavity side, in the treatment method according to the first embodiment;

FIG. 2D is a cross-sectional view showing the false esophagus and the stomach as seen along a line IID-IID of FIG. 2C;

FIG. 3A is a perspective view showing a distal portion of an endoscope apparatus forming the false esophagus, employed for the treatment method according to the first embodiment;

FIG. 3B is a side view showing a distal-side portion of the endoscope apparatus;

FIG. 3C is a longitudinal sectional view showing a modified example of a distal hood of the endoscope apparatus;

FIG. 4A is an illustration showing formation of the false esophagus, in the treatment method according to the first embodiment;

FIG. 4B is an illustration showing a clip member retained and walls of stomach joined by the formation of the false esophagus;

FIG. 4C is an illustration showing the formed false esophagus;

FIG. 5A is an illustration showing engaging the distal portion of the endoscope with the inner surface of the fundus of stomach in making a portion of the fundus of stomach on the peritoneal cavity side approach a portion of the false esophagus on the peritoneal cavity side, in the treatment method according to the first embodiment;

FIG. 5B is an illustration showing contacting the portion of the fundus of stomach on the peritoneal cavity side and a portion of the false esophagus on the peritoneal cavity side in the approach;

FIG. 6A is a perspective view showing a distal portion of an endoscope apparatus fixing a portion of the fundus of stomach and a portion of the false esophagus which are made to approach to each other, as employed in the treatment method according to the first embodiment;

FIG. 6B is a perspective view showing a distal portion of a grasping forceps of the endoscope apparatus;

FIG. 6C is a longitudinal sectional view showing a distal portion of a puncturing needle of the endoscope apparatus;

FIG. 6D is a perspective view showing a proximal portion of the endoscope apparatus;

FIG. 6E is a longitudinal sectional view showing a mechanism which operates the puncturing needle of the endoscope apparatus;

FIG. 7A is a side view showing an opened grasping forceps, in the fixation of a portion of the fundus of stomach and a portion of the false esophagus which are made to approach to each other, in the treatment method according to the first embodiment;

FIG. 7B is an illustration showing puncturing of a puncturing needle through a body wall and pushing out of a first T bar, in the fixation;

FIG. 7C is an illustration showing a retained member retained and the body wall fixed, in the fixation;

FIG. 8A is an illustration showing an initial state of winding the fundus of stomach around the peritoneal cavity side of the false esophagus, in the treatment method according to the first embodiment;

FIGS. 8B and 8C are illustrations showing winding the fundus of stomach around the peritoneal cavity side of the false esophagus, in the treatment method according to the first embodiment;

FIG. 9 is an illustration showing a modified example of winding the fundus of stomach around the peritoneal cavity side of the false esophagus, in the treatment method according to the first embodiment;

FIG. 10 is an illustration showing the esophagus and the stomach treated in the treatment method according to the first embodiment;

FIG. 11 is a perspective view showing a distal portion of an endoscope apparatus forming a false esophagus, in a treatment method according to a second embodiment of the present invention;

FIG. 12A is an illustration showing a first prong of a clip member sticking into an anterior wall of stomach, in the formation of the false esophagus, in the treatment method according to the second embodiment;

FIG. 12B is an illustration showing a second prong of the clip member sticking into a posterior wall of stomach, in the formation of the false esophagus;

FIG. 13A is a perspective view showing a distal portion of an endoscope apparatus forming a false esophagus, employed for a treatment method according to a third embodiment of the present invention;

FIG. 13B is an illustration showing a binding forceps and a retaining member of the endoscope apparatus;

FIG. 13C is a front view showing a distal hood of the endoscope apparatus;

FIG. 14A is an illustration showing a first clip portion engaged with an anterior wall of stomach and a second clip portion engaged with a posterior wall of stomach, in the formation of the false esophagus, in the treatment method according to the third embodiment of the present invention;

FIG. 14B is an illustration showing reduction of a distance between the first and second clip portions, in the formation of the false esophagus;

FIG. 15A is a side view showing a distal portion of an endoscope apparatus forming a false esophagus, in a treatment method according to a fourth embodiment of the present invention;

FIG. 15B is an illustration showing a distal portion of a puncturing needle and a distal portion of a binding forceps, of the endoscope apparatus;

FIG. 15C is an illustration showing a retained member and the binding forceps of the endoscope apparatus;

FIG. 15D is an illustration showing a fastening member of the retained member of the endoscope apparatus;

FIGS. 15E and 15F are illustrations showing an operation of an urging member in the fastening member of the retained member of the endoscope apparatus;

FIG. 16 is an illustration showing a treatment method according to the fourth embodiment of the present invention;

FIG. 17A is a side view showing an endoscope apparatus forming a false esophagus, in a treatment method according to a fifth embodiment of the present invention;

FIG. 17B is a side view showing a distal portion of an overtube in the endoscope apparatus;

FIG. 17C is an exploded view showing a proximal portion of the overtube in the endoscope apparatus;

FIG. 18A is an illustration showing an endoscope inserted into a stomach, in the formation of the false esophagus, in the treatment method according to the fifth embodiment of the present invention;

FIG. 18B is a longitudinal sectional view showing an anterior wall of stomach and a posterior wall of stomach which are sucked into the overtube and stuck by a puncturing needle in the overtube, in the formation of the false esophagus;

FIG. 18C is a transverse sectional view showing an anterior wall of stomach and a posterior wall of stomach which are sucked into the overtube and stuck by a puncturing needle in the overtube, in the formation of the false esophagus;

FIG. 18D is a longitudinal sectional view showing a retained member, in the formation of the false esophagus;

FIG. 18E is an illustration showing the false esophagus, in the formation thereof;

FIG. 19A is an illustration showing abrasion of a mucous membrane in the formation of the false esophagus, in a treatment method according to a sixth embodiment of the present invention;

FIG. 19B is an illustration showing a retained clip portion and joined walls of stomach, in the treatment method according to the sixth embodiment;

FIG. 20A is an illustration showing winding a fundus of stomach around a peritoneal cavity side of a false esophagus, in a treatment method according to a seventh embodiment of the present invention; and

FIG. 20B is an illustration showing fixing the wound fundus of stomach and the peritoneal cavity side of the false esophagus, in the treatment method according to the seventh embodiment.

### DETAILED DESCRIPTION OF THE INVENTION

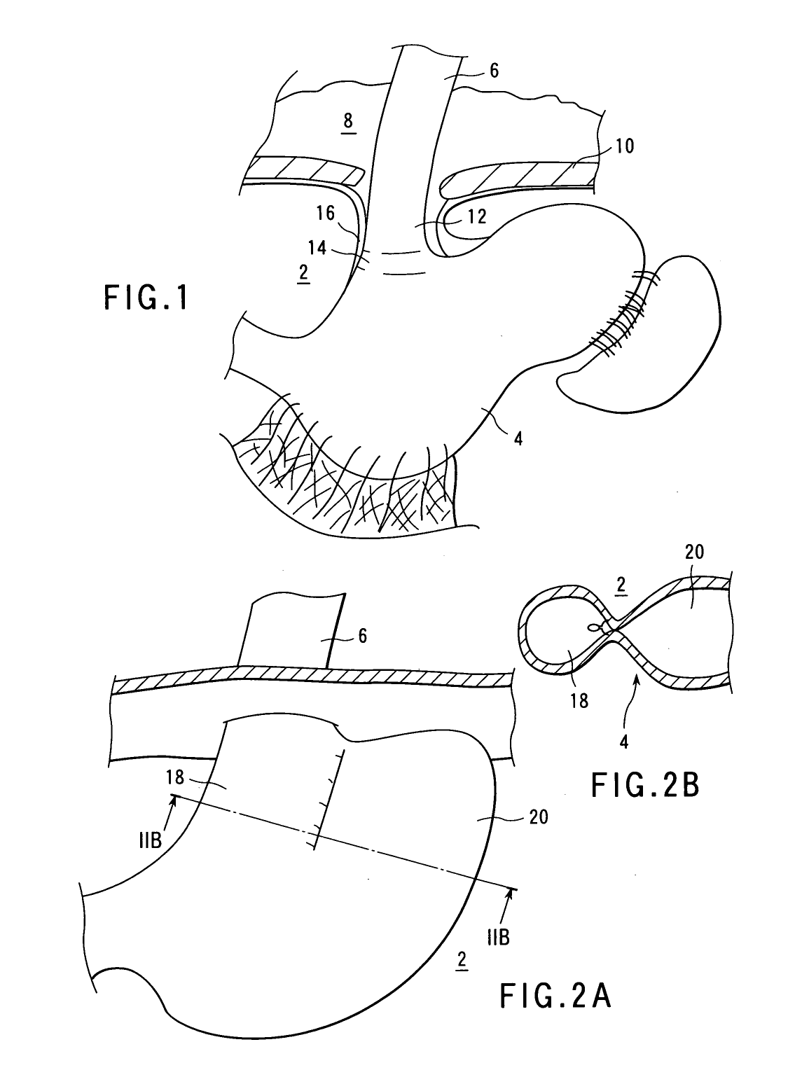

A first embodiment of the present invention will be described with reference to FIGS. 1 to 10. FIG. 1 anatomically shows a periphery of a stomach 4 and an esophagus 6 in a peritoneal cavity 2. The esophagus 6 extends from the inside of a thoracic cavity 8 into the peritoneal cavity 2 through a gap of a diaphragm 10 and connects with the stomach 4. A part of the esophagus 6 located inside the peritoneal cavity 2 is called an abdominal esophagus 12 and a boundary between the esophagus 6 and the stomach 4 is called a gastroesophageal boundary 14. The peritoneal cavity 2 sides of the diaphragm 10, the abdominal esophagus 12, and the gastroesophageal boundary 14 are covered with a peritoneum 16. In other words, the abdominal esophagus 12 is hardly exposed to the peritoneal cavity 2.

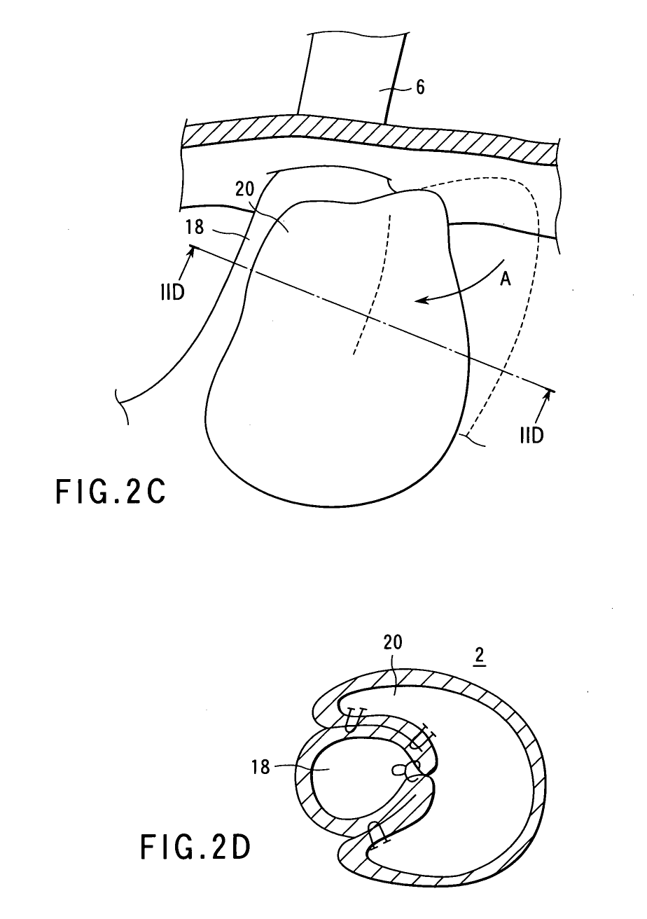

An outline of the treatment method according to the first embodiment of the present invention will be described with reference to FIGS. 2A to 2D. First, a false esophagus 18 is formed by joining an inner surface of an anterior wall of stomach and an inner surface of a posterior wall of stomach to each other so as to extend the original esophagus 6 as shown in FIG. 2A. The stomach is divided into two areas with the joined part as shown in FIG. 2B. One of the areas is shaped in a cylinder which communicates with a lower end of the esophagus 6 and which has substantially the same inner diameter as the esophagus 6. This cylindrical area is the false esophagus 18. A fundus of stomach 20 exists in the other area.

After the false esophagus 18 is formed, the fundus of stomach 20 is wound round the false esophagus 18 from a peritoneal cavity side as represented by an arrow A of FIG. 2C. The fundus of stomach 20 is fixed on the false esophagus 18 to maintain the state of being wound round the false esophagus 18 as shown in FIG. 2D.

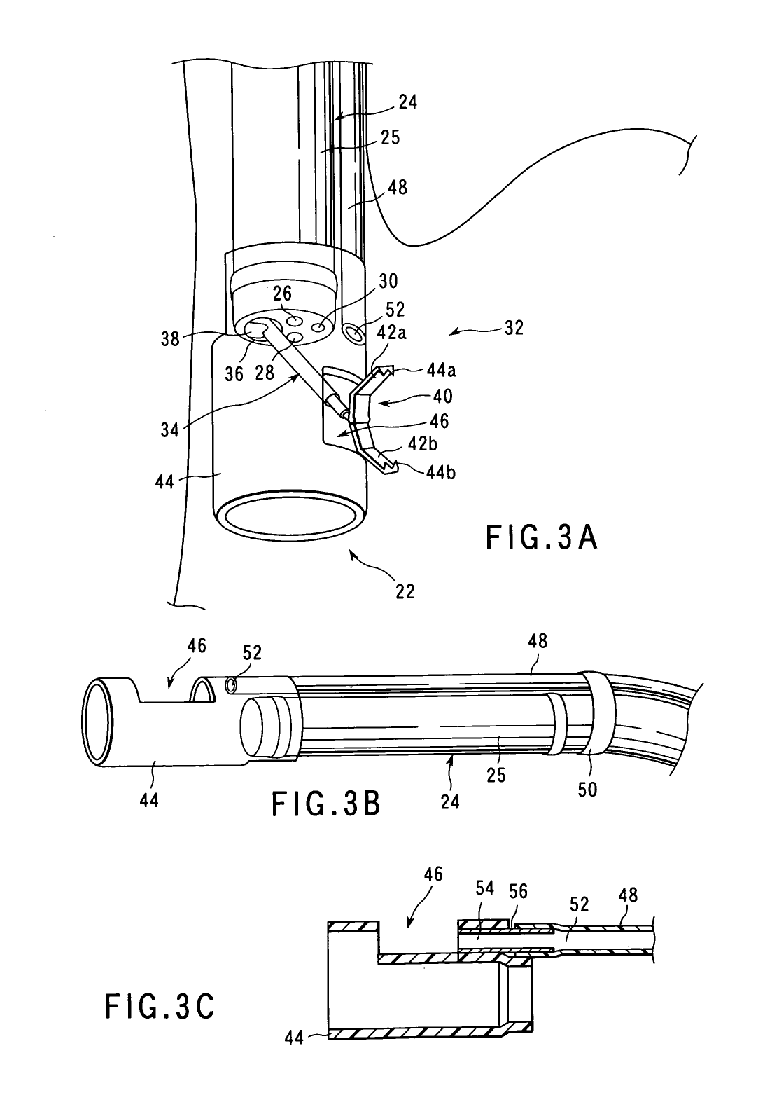

Next, the treatment method of this embodiment will be described more specifically with reference to FIGS. 3A to 10. An endoscope apparatus 22 of this embodiment to form the false esophagus 18 is described with reference to FIGS. 3A to 3C. The endoscope apparatus 22 has an endoscope 24 as shown in FIG. 3A. The endoscope 24 is inserted into a body cavity for observation and has a channel through which a treatment instrument is inserted.

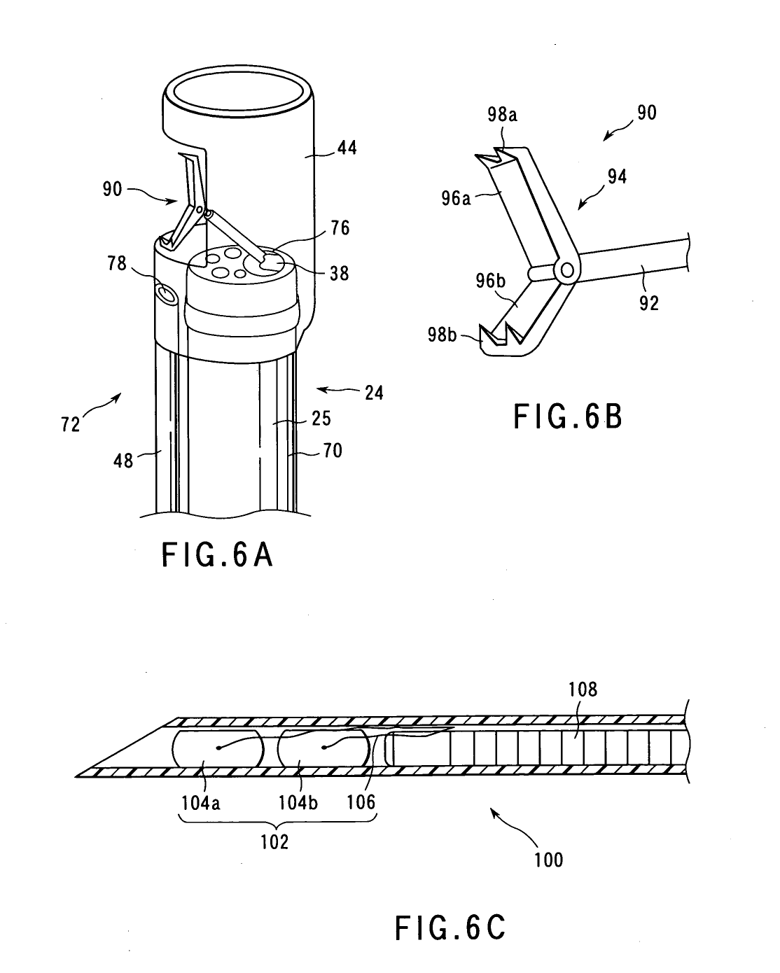

The endoscope 24 is an electronic endoscope or a fiber-optic endoscope. The endoscope 24 also has an insertion portion 25 which is perorally inserted into the stomach. A lens 26 for observation, a lens 28 for illumination and a nozzle 30 for cleaning are arranged at a distal end surface of the insertion portion 25, similarly to a general endoscope. An operation portion 110 (see FIG. 6D) rotating the endoscope 24 about a central axis of the endoscope 24 is provided at a proximal portion of the endoscope 24.

A forceps channel 36 is formed at the insertion portion 25. The forceps channel 36 extends from the proximal portion of the endoscope 24 to the distal portion thereof and has an opening on the distal end surface of the insertion portion 25. A clip applicator 34 as a retaining forceps is inserted into the forceps channel 36. An elevator 38 as adjusting member capable of adjusting a direction of the clip applicator 34 projecting from the forceps channel 36 is arranged in the vicinity of the opening portion of the forceps channel 36.

A clip member 40 as a member to be retained is provided at a distal portion of the clip applicator 34. The clip member 40 has a first grasping portion 42a and a second grasping portion 42b that are configured to relatively open or close. The first and second grasping portions 42a and 42b have scales capable of simultaneously grasping the anterior wall and the posterior wall which are pulled toward each other as explained later when the grasping portions 42a and 42b are opened at the maximum. First and second prongs 44a and 44b as first and second engagement portions that stick in the walls of stomach are provided on inner side surfaces of distal portions of the first and second grasping portions 42a and 42b, respectively. The first and second prongs 44a and 44b are long enough to reach a muscularis of gastric tissues when they stick in the walls of stomach. The clip member 40 is opened and closed, moved or retained by the clip applicator 34 on the basis of operations on the proximal side of the endoscope 24.

A distal hood 44, which is a tube-like member, is arranged at the distal portion of the insertion portion 25. The distal portion of the insertion portion 25 is fitted in a proximal portion of the distal hood 44. The distal hood 44 may be formed integrally with the insertion portion 25. The distal hood 44 has sufficient strength to prevent from being crushed when the interior of the distal hood 44 is sucked as explained later. A side aperture 46 is formed to an outer peripheral wall of the distal hood 44. The side aperture 46 serves as a communication portion which makes the inner and outer sides of the distal hood 44 communicate with each other. The clip applicator 34 can project from the inner side of the distal hood 44 to the outer side thereof through the side aperture 46.

A sheath 48 is provided at the outer peripheral surface of the endoscope 24, along an axial direction of the insertion portion 25 of the endoscope 24. The sheath 48 is inserted into the distal hood 44 from the proximal side of the distal hood 44 and an outer peripheral surface of the sheath 48 is fixed on the inner peripheral surface of the distal hood 44. A distal portion of the sheath 48 is arranged on the proximal side from the side aperture 46 and aligned with a central portion of the side aperture 46 in the longitudinal direction of the distal hood 44. The sheath 48 is fixed on the endoscope 24 by a tape 50 or the like as shown in FIG. 3B. A suction channel 52 sucking the interior of the distal hood 44 is formed by an inner cavity of the sheath 48.

The sheath 48 may be capable of being freely attached to or removed from the distal hood 44 as shown in FIG. 3C. That is, a distal channel 54 is formed integrally with the distal hood 44, on the proximal side of the side aperture 46. The distal channel 54 extends in a direction of a central axis of the distal hood 44. A cap 56 projects from a rear side of the distal channel 54. An end portion of the sheath 48 is mounted on the cap 56 so as to be freely detached therefrom. The suction channel 52 may be formed on the endoscope 24.

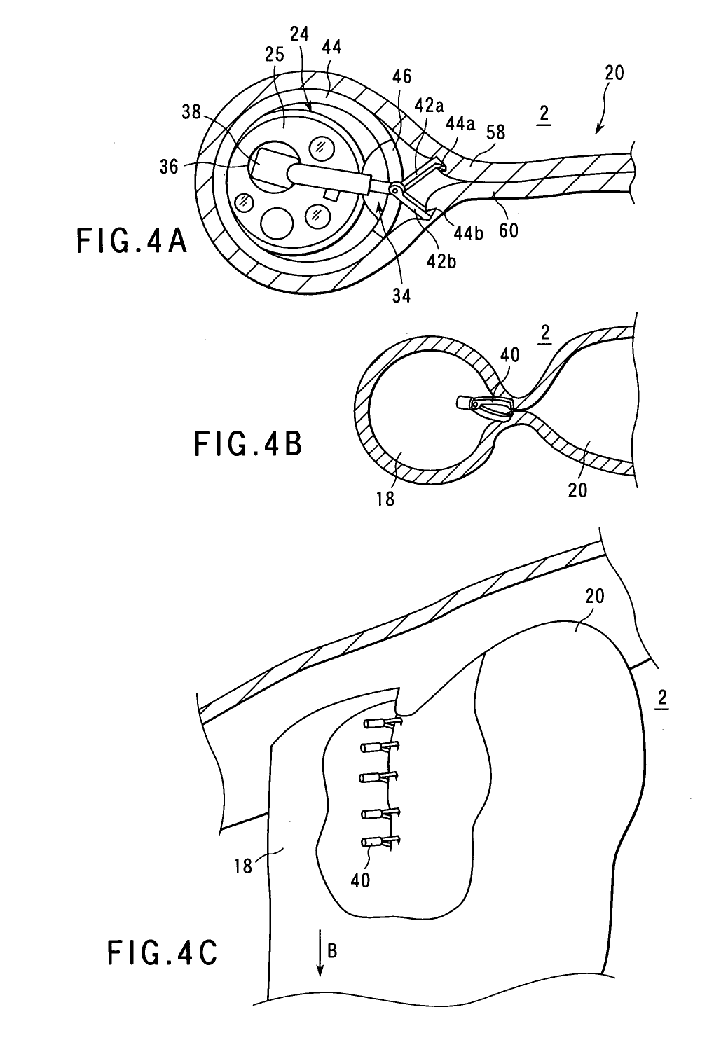

Forming the false esophagus 18 by using the endoscope apparatus 22 will be explained with reference to FIGS. 4A to 4C. The endoscope apparatus 22 is perorally inserted into the esophagus 6 and the distal portion of the endoscope apparatus 22 projects inside the stomach. The distal hood 44 is arranged at a position where the false esophagus 18 is to be formed. Furthermore, the endoscope 24 is operated to rotate about the central axis of the endoscope 24, and the side aperture 46 is arranged between an anterior wall of stomach 58 and a posterior wall of stomach 60 to face the fundus of stomach 20. After that, the interior of the distal hood 44 is sucked via the suction channel 52. As a result, the stomach shrinks. The anterior wall of stomach 58 clings around one side of the outer peripheral surface of the distal hood 44 and the posterior wall of stomach 60 clings around the other side thereof as shown in FIG. 4A. Thus, the inner surfaces of the anterior wall of stomach 58 and the posterior wall of stomach 60 are mutually pulled toward each other to make a contact in the vicinity of the side aperture 46 of the distal hood 44.

In this state, the clip applicator 34 is inserted into the forceps channel 36 of the endoscope 24. The direction of advance of the clip applicator 34 is adjusted by the elevator 38. The clip applicator 34 is pushed toward the side aperture 46 to project to the outside of the distal hood 44 through the side aperture 46. The clip member 40 is operated by the clip applicator 34 to join the anterior wall of stomach 58 and the posterior wall of stomach 60 which are pulled toward each other. That is, the first prong 44a is stuck and engaged with the anterior wall of stomach 58 and the second prong 44b is stuck and engaged with the posterior wall of stomach 60. The first and second grasping portions 42a and 42b are relatively closed. After that, the clip member 40 is retained while remaining closed as shown in FIG. 4B. At this time, an adhesive may be applied to the junction to help joining.

The clip member 40 is retained at some positions spaced in a direction parallel to the axis of the esophagus, from the fundus of stomach 20 side of the gastroesophageal boundary 14 to the anal side, (arrow B of FIG. 4C). As a result, the cylindrical false esophagus 18 is formed by the wall of stomach on a lesser curvature side, the anterior wall of stomach, the posterior wall of stomach, and junction formed by the clip member 40 as shown in FIG. 4C. The false esophagus 18 has substantially the same inner diameter as the esophagus 6 and extends the esophagus 6 about a few centimeters or ten and a few centimeters from the cardiac region to the interior of the stomach.

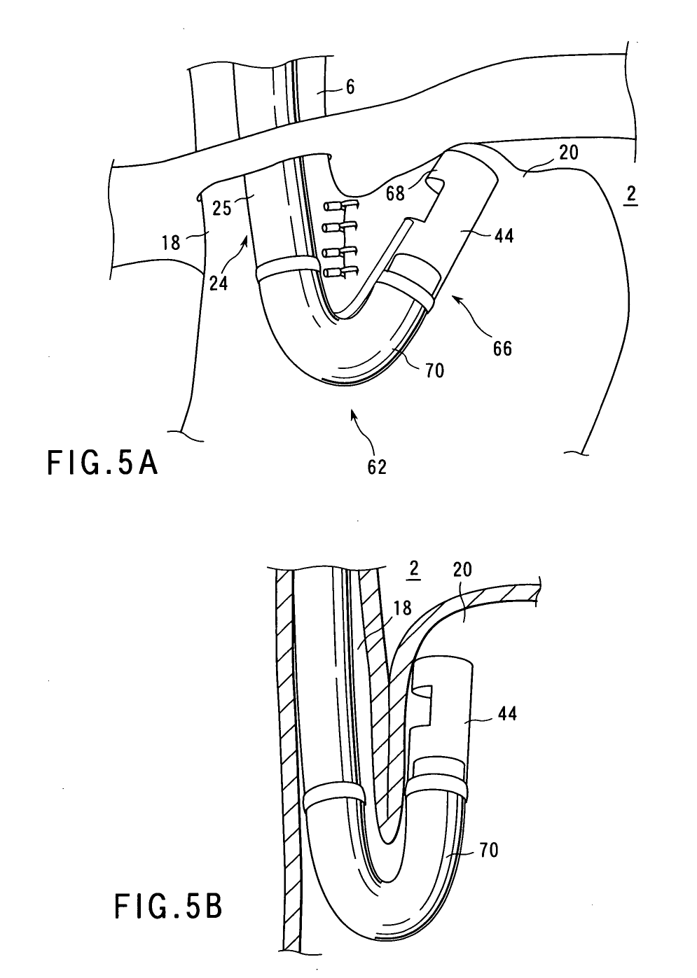

Next, an endoscope apparatus 62 of this embodiment making a portion of the fundus of stomach 20 on the peritoneal cavity 2 side approach a portion of the false esophagus 18 on the peritoneal cavity 2 side will be described with reference to FIG. 5A. The endoscope apparatus 62 has the endoscope 24. The endoscope 24 is inserted into the body cavity for observation and has a bending portion 70 operated to bend.

The distal hood 44 is provided at the distal portion of the insertion portion 25 of the endoscope 24. The proximal portion of the distal hood 44 is fitted in the distal portion of the insertion portion 25. The distal hood 44 may be formed integrally with the distal portion of the insertion portion 25. An engaging portion 68 configured to engage with the inner surface of the fundus of stomach 20 is formed at the distal portion of the distal hood 44. A bending portion 70 operated to be bent by the operation portion 110 is provided at the insertion portion 25.

The length and shape of the distal hood 44 are set in accordance with the shape of the stomach peculiar to each patient such as the distance between the esophagus 6 and the fundus of stomach 20, the length of the false esophagus 18, and the like, such that the part of the peritoneal cavity 2 side of the fundus of stomach 20 approach the part of the peritoneal cavity 2 side of the false esophagus 18 as explained later.

The endoscope apparatus 62 can be configured to have a function of forming the false esophagus 18 and a function of making the part of the peritoneal cavity 2 side of the fundus of stomach 20 approach the part of the peritoneal cavity 2 side of the false esophagus 18. The endoscope apparatus 62 having these two functions can be formed by adding the engaging portion 68 and the bending portion 70 to the endoscope apparatus 22 (FIG. 3A) for forming the false esophagus 18, as shown in FIG. 5A. In the endoscope apparatus 62 for forming the false esophagus 18, the bending portion 70 is not definitely required. However, if the bending portion 70 is provided, the false esophagus 18 can be formed more easily.

Making a portion of the fundus of stomach 20 on the peritoneal cavity 2 side approach a portion of the false esophagus 18 on the peritoneal cavity 2 side, with the endoscope apparatus 62, will be described with reference to FIGS. 5A and 5B. The endoscope 24 is perorally inserted into the esophagus 6 and further into the false esophagus 18. The distal portion of the endoscope 24 projects into the stomach beyond the false esophagus 18. After that, the bending portion 70 is bent such that the distal portion of the distal hood 44 can turn at the junction of the clip member 40 and move from the lesser curvature side to the fundus of stomach 20. The engaging portion 68 is engaged with the inner surface of the fundus of stomach 20 as shown in FIG. 5A. The bending portion 70 is further bent with the engaging portion 68 engaged with the inner surface of the fundus of stomach 20. As a result, a portion of the fundus of stomach 20 on the peritoneal cavity 2 side is made to approach and contact a portion of the false esophagus 18 on the peritoneal cavity 2 side as shown in FIG. 5B.

Next, an endoscope apparatus 72 of this embodiment mutually fixing a portion of the fundus of stomach 20 and a portion of the false esophagus 18 which are made to approach, will be described with reference to FIGS. 6A to 6E. The endoscope apparatus 72 has the endoscope 24 as shown in FIG. 6A. The endoscope 24 is inserted into the body cavity for observation and has a bending portion 70 operated to bend and the channel through which a treatment instrument is inserted.

The endoscope apparatus 72 has the distal hood 44, a first forceps channel 76, the elevator 38, and a second forceps channel 78, similarly to the distal hood 44, the forceps channel 36, the elevator 38, and the suction channel 52 (FIG. 3A) in the endoscope apparatus 22 forming the false esophagus. The endoscope apparatus 72 has the bending portion 70, similarly to the bending portion 70 of the endoscope apparatus 22. Moreover, similarly to the distal hood 44 of the endoscope apparatus 22, the length and shape of the distal hood 44 are set in accordance with the shape of the stomach peculiar to each patient such as the distance between the esophagus 6 and the fundus of stomach 20, the length of the false esophagus 18, and the like, such that the fundus of stomach 20 and the false esophagus 18 can be fixed as explained later.

A grasping forceps 90 as a pulling forceps which pulls a portion of the fundus of stomach 20 on the peritoneal cavity side and a portion of the false esophagus 18 on the peritoneal cavity side while they are stacked as explained later, can be inserted into the first forceps channel 76. A first grasping portion 96a and a second grasping portion 96b which are relatively opened or closed are provided at a distal portion of the grasping forceps 90. The first and second grasping portions 96a and 96b are large enough to simultaneously grasp the body walls of two layers as explained later. First and second sharp portions 98a and 98b project from inner side surfaces of the distal portions of the first and second grasping portions 96a and 96b, respectively. A grasping sheath 92 extends from the first and second grasping portions 96a and 96b. The first and second grasping portions 96a and 96b are supported to be rotatable at a distal portion of the grasping sheath 92. So, the first and second grasping portions 96a and 96b can be rotated to the distal portion of the grasping sheath 92.

Thus, the opening and closing mechanism of the grasping forceps 90 is passive. Of course, the opening and closing mechanism of the grasping forceps 90 may be active and can be operated to open or close, on the proximal side, by the operator.

On the other hand, a puncturing needle 100 as a needle-shaped forceps shown in FIG. 6C can be inserted into the second forceps channel 78. The puncturing needle 100 has an inner cavity whose vertical section in the lengthwise direction of the puncturing needle 100 is substantially shaped in a circle. A retained member 102 which is to be retained in the stomach is contained in the distal portion of the inner cavity. The retained member 102 has first and second T-bars 104a and 104b as first and second rod-like members. The first and second T-bars 104a and 104b are shaped in a column and are sequentially fitted in the inner cavity. The first and second T-bars 104a and 104b are connected to each other with a thread 106 as a thread-like member.

A pusher 108 as a pushing member which discharges the first and second T bars 104a and 104b from the distal portion of the puncturing needle 100 to the outside is contained in a proximal side of the inner cavity. The pusher 108 extends to a proximal end inside the inner cavity.

The sheath 48 forming the second forceps channel 78 (FIG. 6A) is fixed at the operation member 110 of the endoscope 24 or a snap preventing member 114 which connects the operation member 110 and an insertion portion 25, with a tape 50 or the like, as shown in FIG. 6D. A sheath handle 116 which is to be held by the operator is provided at the proximal portion of the sheath 48. A needle handle 118 operating advance and retreat of the puncturing needle 100 is provided at a proximal portion of the sheath handle 116. A pusher handle 120 operating advance and retreat of the pusher 108 is provided a proximal portion of the needle handle 118.

The sheath handle 116 is substantially shaped in a cylinder, and the proximal end surface of the sheath 48 is connected to the distal end surface of the sheath handle 116 as shown in FIG. 6E. The needle handle 118 is substantially shaped in a cylinder and a distal side portion thereof is fitted in the inner cavity of the sheath handle 110 from the proximal side so as to freely advance or retreat. A proximal side portion of the needle handle 118 has a greater outer diameter than a proximal side portion thereof and is held by the operator. The puncturing needle 100 is inserted into the sheath handle 116 and a proximal end of the puncturing needle 100 is connected to a distal end of the needle handle 118. A distal side portion of the pusher handle 120 has a columnar shape and is fitted in the inner cavity of the sheath handle 116 from the proximal side so as to freely advance or retreat. A pushing portion 121 which is pushed by the operator is provided at a proximal side portion of the pusher handle 120. The puncturing needle 100 and the needle handle 118 are inserted into the pusher 108. A proximal portion of the pusher 108 is coupled to a distal portion of the pusher handle 120.

The endoscope apparatus 72 can be configured to have a function of forming the false esophagus 18, a function of making the part of the peritoneal cavity 2 side of the fundus of stomach 20 approach the part of the peritoneal cavity 2 side of the false esophagus 18, and a function of fixing the part of the fundus of stomach 20 and the part of the false esophagus 18 which are made to approach. As explained above, the endoscope apparatus 62 can be configured to have a function of forming the false esophagus 18 and a function of making the part of the peritoneal cavity 2 side of the fundus of stomach 20 approach the part of the peritoneal cavity 2 side of the false esophagus 18 (FIG. 5A). The endoscope apparatus 72 having these three functions can be formed by allowing the puncturing needle 100 (FIG. 6C) to be inserted into the suction channel 52 (FIG. 3A) in the endoscope apparatus 62 having the two functions.

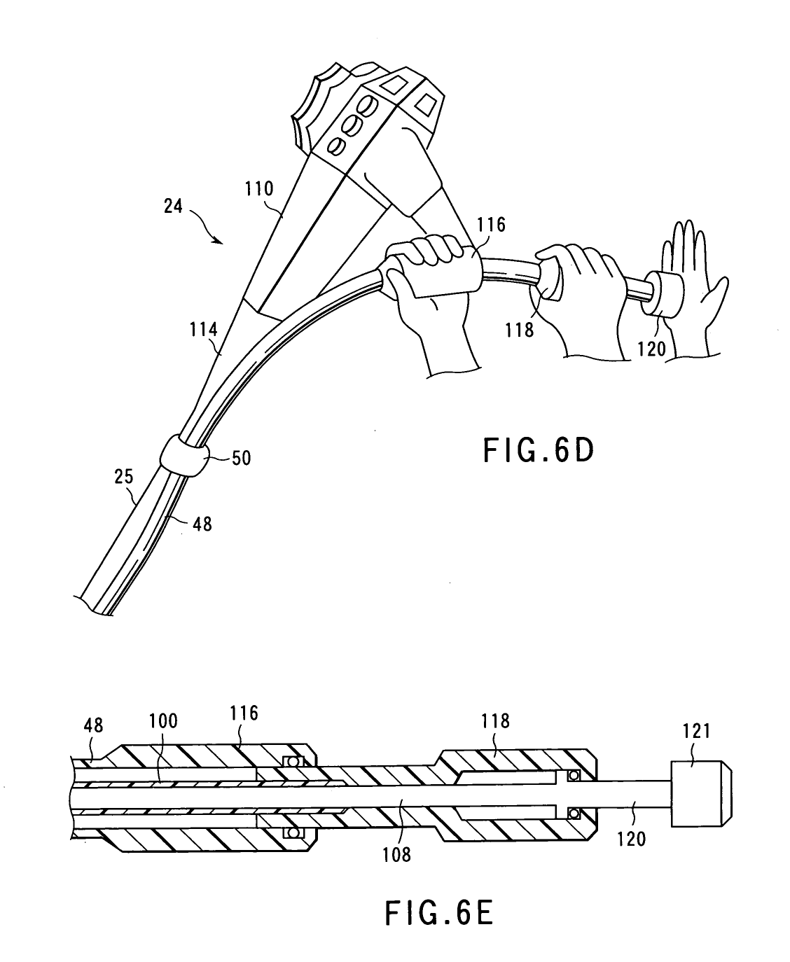

Next, mutual fixation of a portion of the fundus of stomach 20 and a portion of the false esophagus 18 which are made to approach, will be described with reference to FIGS. 7A to 7C. The grasping forceps 90 projects from the first forceps channel 76 while a portion of the body wall of the fundus of stomach 20 and a portion of the body wall of the false esophagus 18 are stacked (FIG. 5B). The grasping forceps 90 is pushed toward the side aperture 46 of the distal hood 44 to project from the side aperture 46 (FIG. 6A). At this time, the first grasping portion 96a of the grasping forceps 90 is elongated in a direction of extending the grasping sheath 92. The first and second grasping portions 96a and 96b are opened asymmetrically to the grasping sheath 92.

The grasping forceps 90 is further pushed, and the first grasping portion 96a is pressed against the inner surface of the fundus of stomach 20 to be grasped and moved in the opening direction. The first and second grasping portions 96a and 96b are pushed by the inner surface of the fundus of stomach 20 and opened. The grasping forceps 90 is further pushed, and the first and second sharp portions 98a and 98b stick and engage with the inner surface of the fundus of stomach 20.

While the first and second sharp portions 98a and 98b keep sticking in the inner surface of the fundus of stomach 20, the grasping forceps 90 pulled toward the proximal side and moved in a direction of going away from the false esophagus 18 by the elevator 38. As a result, the first and second grasping portions 96a and 96b are closed, and the portion of the body wall of the fundus of stomach 20 and the portion of the body wall of the false esophagus 18 are grasped with they being stacked, by the grasping forceps 90. The grasping forceps 90 is further pulled toward the proximal side and moved in the direction of going away from the false esophagus 18 by the elevator 38. The body wall of the fundus of stomach 20 and the body wall of the false esophagus 18 are pulled into the distal hood 44 with they being stacked as shown in FIG. 7B.

After that, the needle handle 118 and the pusher handle 120 are moved integrally to the distal side relative to the sheath handle 116. As a result, the puncturing needle 100 projects from the sheath 48, is pushed, is inserted into the false esophagus 18 through the body walls of the fundus of stomach 20 and the false esophagus 18, and then inserted into the stomach through the body walls of the false esophagus 18 and the fundus of stomach 20.

In this state, the pusher handle 120 is moved to the distal side relative to the needle handle 118. As a result, the pusher 108 pushes the first and second T-bars 104a and 104b to the distal side, and discharges the first T-bar 104a from the puncturing needle 100 into the stomach. After that, the needle handle 118 and the pusher handle 120 are moved integrally to the proximal side relative to the sheath handle 116. As a result, the puncturing needle 100 is extracted sequentially from the body walls. The second T-bar 104b is discharged from the puncturing needle 100 into the stomach.

After that, the grasping forceps 90 is pushed, and moved in a direction of approaching the false esophagus 18 by the elevator 38. The body walls of the fundus of stomach 20 and the false esophagus 18 are returned to their initial positions. The grasping forceps 90 is further pushed, and the first and second grasping portions 96a and 96b are pushed and opened by the inner surface of the fundus of stomach 20. As a result, the sharp portions 98a and 98b are extracted from the body wall of the fundus of stomach 20. After that, the grasping forceps 90 is pulled to the proximal side and contained into the distal hood 44. Thus, the portion of the fundus of stomach 20 on the peritoneal cavity side and the portion of the false esophagus 18 on the peritoneal cavity side are mutually fixed as shown in FIG. 7C.

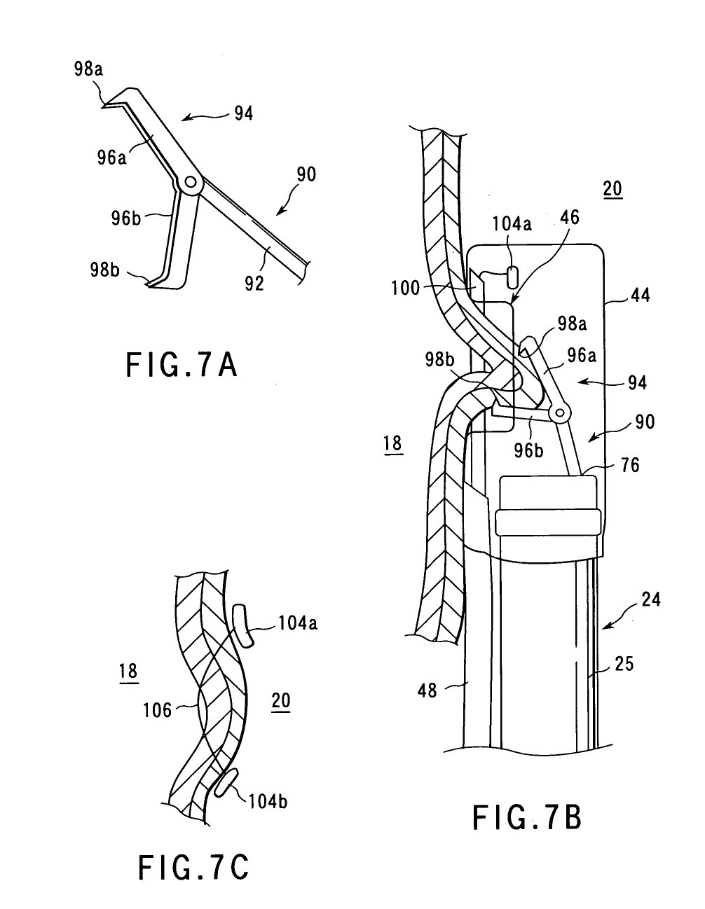

Next, winding the fundus of stomach 20 around the peritoneal cavity side of the false esophagus 18 will be described with reference to FIGS. 8A to 8C. The endoscope apparatus 62 for the approaching can be employed as the endoscope apparatus for the winding.

To wind the fundus of stomach 20 around the peritoneal cavity side of the false esophagus 18, first, the fundus of stomach 20 and the false esophagus 18 are made to approach and fixed in the vicinity of the junction of the anterior wall and the posterior wall, as shown in FIG. 8A. After that, the operation member 110 (FIG. 6D) is operated such that the endoscope 24 is rotated about its own central axis. As a result, the distal hood 44 at the distal portion of the endoscope 24 is rotated around the central axis of the false esophagus 18. The distal hood 44 is moved in a distance in a peripheral direction of the false esophagus 18 and arranged as shown in FIG. 8B. At this position, a portion of the fundus of stomach 20 and a portion of the false esophagus 18 are mutually fixed, similarly to the above fixation.

Subsequently, the endoscope 24 is pushed or pulled such that the distal hood 44 is moved in the axial direction of the false esophagus 18, from the vicinity of the cardiac region to the center of the stomach or from the center of the stomach to the cardiac region. Thus, the portion of the fundus of stomach 20 on the peritoneal cavity side and the portion of the false esophagus 18 on the peritoneal cavity side are mutually fixed, at a plurality of positions spaced from each other in the axial direction of the false esophagus 18.

Subsequently, the endoscope 24 is rotated in a direction opposite to that of the winding operation, around its own central axis. As a result, the distal hood 44 at the distal portion of the endoscope 24 is rotated in the direction opposite to that of the winding direction around the central axis of the false esophagus 18. The distal hood 44 is arranged at a position symmetrical with the position arranged in the above operation, relative to the junction of the false esophagus 18 as shown in FIG. 8C. The fixing operation is executed at this position, similarly to the above-described fixing operation.

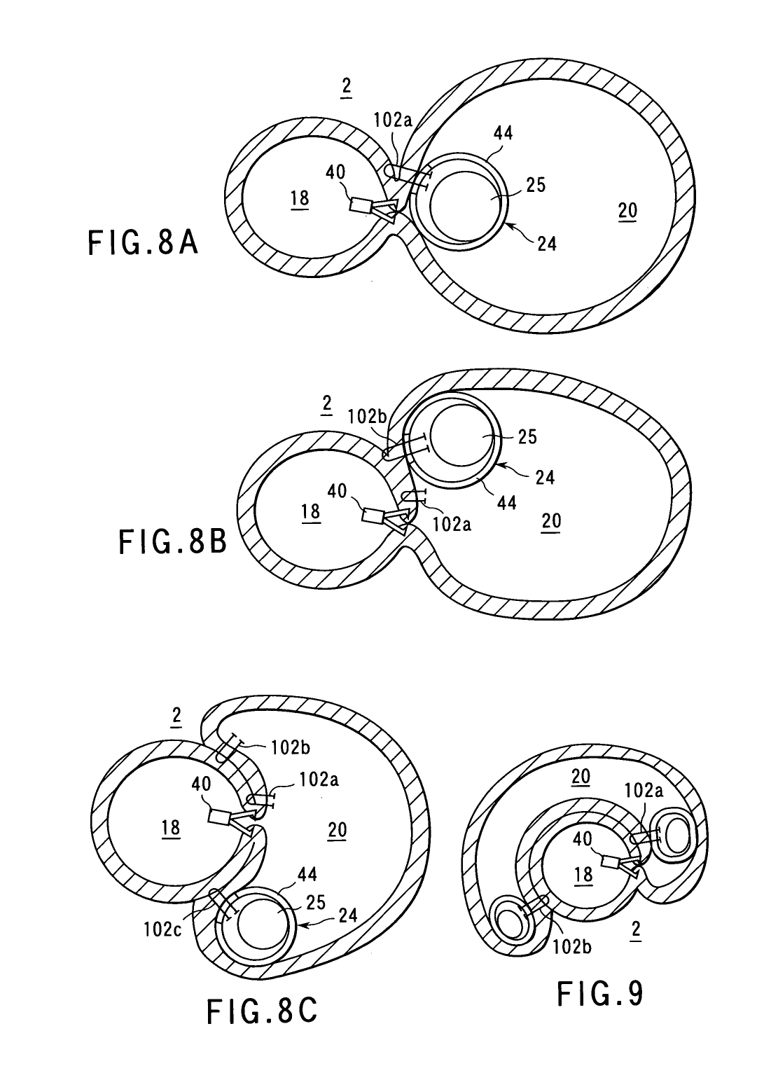

In this embodiment, first, a portion of the fundus of stomach 20 and a portion of the false esophagus 18 are mutually fixed, in the vicinity of the junction of the false esophagus 18. However, the initial fixation may not be executed. In addition, the fundus of stomach 20 may not be wound on both sides of the false esophagus 18, but wound on either side thereof and fixed as shown in FIG. 9.

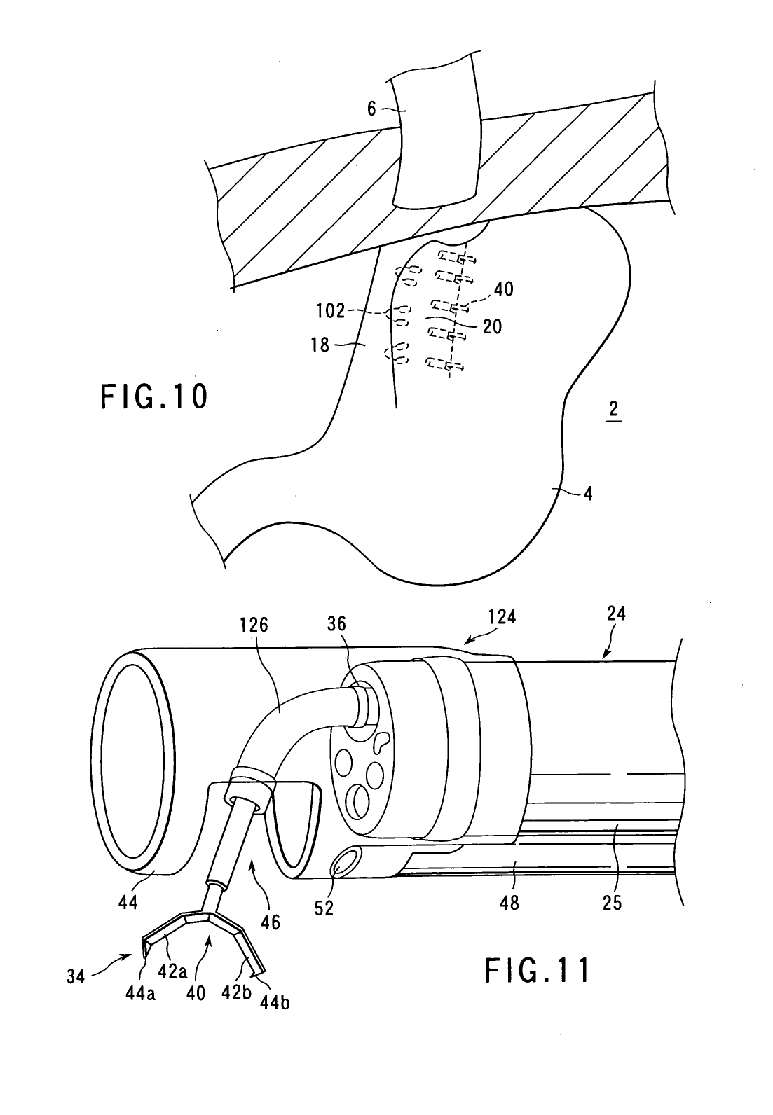

FIG. 10 illustrates a completed form of the stomach 4 and the esophagus 6 treated in the treatment method according to the first embodiment of the present invention. When the fundus of stomach 20 wound round the false esophagus 18 expands, the false esophagus 18 is pressed and closed. Thus, the cardiac region is reconstituted and the reflux is prevented.

In this embodiment, the false esophagus 18 is formed inside the stomach by perorally pulling the anterior wall of stomach 58 and the posterior wall of stomach 60 toward each other, and joining a portion of the inner surface of the anterior wall of stomach 58 and that of the posterior wall of stomach 60 to extend the esophagus 6. The fundus of stomach 20 is perorally wound round the peritoneal cavity side of the false esophagus 18. The portion of the fundus of stomach 20 and the portion of the false esophagus 18 are mutually fixed so as to maintain the winding state. In other words, all the operations are perorally executed in the treatment method of this embodiment. For this reason, invasion to a patient is small and burden on the patient is reduced.

In addition, the false esophagus 18 is pressed and closed by expanding the fundus of stomach 20 wound round the false esophagus 18. The stroke of expansion and contraction of the fundus of stomach 20 is sufficiently great as compared with the stroke of expansion and contraction of the other portions of the stomach, for example, the gastroesophageal boundary 14 and the vicinity thereof. For this reason, if the treatment method of this embodiment is applied, the false esophagus 18 sufficiently pressed down and the reflux is effectively prevented, as compared with a case where, for example, the other portions of the stomach, for example, the gastroesophageal boundary 14 and the vicinity thereof are wound round the esophagus 6 or the false esophagus 18.

The fundus of stomach 20 is directly wound round the false esophagus 18 and thereby fixed. Anatomically, the peritoneal cavity 2 sides of the diaphragm 10, the abdominal esophagus 12 and the gastroesophageal boundary 14 are covered with the peritonem 16 (FIG. 1). For this reason, in a case where the fundus of stomach 20 is wound round the esophagus 6 of the peritoneal cavity side and thereby fixed, the peritonem 16 intervenes between the fundus of stomach 20 and the esophagus 6. As the peritonem 16 can expand and contract independently of the other tissues, the expansion and contraction of the fundus of stomach 20 are absorbed in the peritonem 16 and are hardly transmitted to the esophagus 6. Therefore, when the fundus of stomach 20 is wound round the false esophagus 18 of the peritoneal cavity side and thereby fixed, according to the treatment method of this embodiment, the false esophagus 18 is sufficiently pressed down and the reflux is effectively prevented as compared with a case where the fundus of stomach 20 is wound round the esophagus 6 of the peritoneal cavity side and thereby fixed.

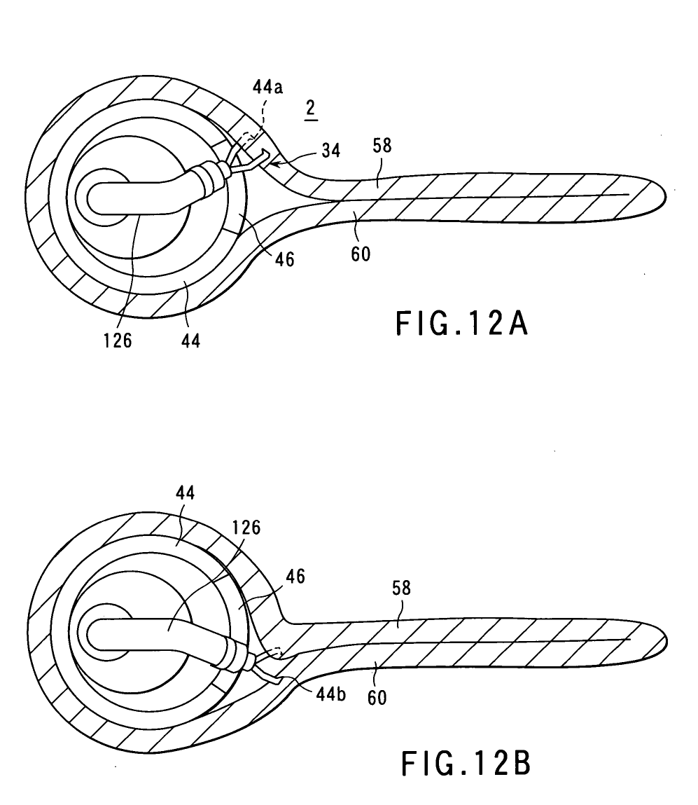

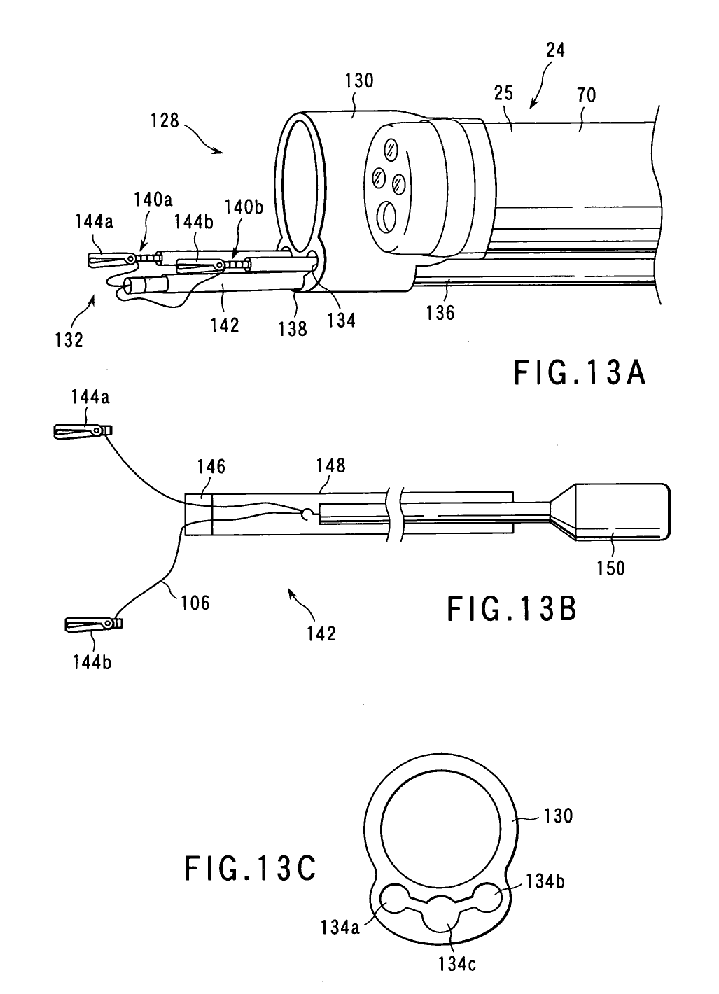

Next, a treatment method according to a second embodiment of the present invention will be described with reference to FIGS. 11 to 12B. The treatment method of this embodiment has a formation of the false esophagus 18, which is different from the formation of the false esophagus 18 in the first embodiment. An endoscope apparatus 124 of this embodiment to form the false esophagus 18 will be described with reference to FIG. 11. The endoscope apparatus 124 is different from the endoscope apparatus 22 (FIG. 3A) of the first embodiment forming the false esophagus, with respect to the only structure explained below.

A bending sheath 126 is inserted into the forceps channel 36. The bending sheath 126 is freely rotatable about its own axis by an operation of the proximal side. A bending portion which can be bent by the operation of the proximal side is provided at a distal portion of the bending sheath 126. The clip applicator 34 can be freely inserted into the bending sheath 126. A elevator is not provided at the forceps channel 36.

Formation of the false esophagus 18 of this embodiment will be described with reference to FIGS. 12A and 12B. As a result of the same operations as those of the first embodiment, the anterior wall of stomach 58 clings around one side of the outer peripheral surface of the distal hood 44 and the posterior wall of stomach 60 clings around the other side thereof as shown in FIG. 12A. In this state, the clip applicator 34 projects from the bending sheath 126.

The clip applicator 34 is made to project from the interior of the distal hood 44 to the outside through the side aperture 46 by operating the clip applicator 34 to advance and retreat or rotate relative to the bending sheath 126 by the operations of the proximal side and bending the bending sheath 126. The first prong 44a of the clip member 40 of the clip applicator 34 sticks and engages with the anterior wall of stomach 58 as shown in FIG. 12A. Subsequently, the clip member 40 is moved toward the posterior wall of stomach 60 and the anterior wall of stomach 58 is pulled toward the posterior wall of stomach 60 with the engagement of the first prong 44a and the anterior wall of stomach 58 being maintained. Thus, the second prong 44b sticks in the posterior wall of stomach 60 as shown in FIG. 12B. After that, the false esophagus 18 is formed by retaining the clip member 40 at some points spaced in a direction parallel to the axis of the esophagus, from the fundus of stomach 20 side of gastroesophageal boundary 14 to the anal side, similarly to the first embodiment.

According to this embodiment, the bending sheath 126 can be bent in which the clip applicator 34 capable of advancing and retreating or rotating relative to the bending sheath 126 is inserted. For this reason, the direction of movement of the clip applicator 34 can be operated by only operating the clip applicator 34 or the bending sheath 126 while maintaining the endoscope 24 at rest. Therefore, the operability is improved.

In addition, the anterior wall of stomach 58 and the posterior wall of stomach 60 can be pulled to each other by operating the clip applicator 34 and the bending sheath 126. For this reason, even if the anterior wall of stomach 58 and the posterior wall of stomach 60 are not sufficiently pulled by suction, the false esophagus 18 can be formed appropriately.

On the other hand, it can be said that the false esophagus 18 can be formed appropriately without sufficiently pulling the anterior wall of stomach 58 and the posterior wall of stomach 60 by suction. In other words, the false esophagus 18 can be formed while sufficiently ensuring a field of view of the endoscope 24 without completely clinging the anterior wall of stomach 58 and the posterior wall of stomach 60 around the distal hood 44. Therefore, the operability is improved.

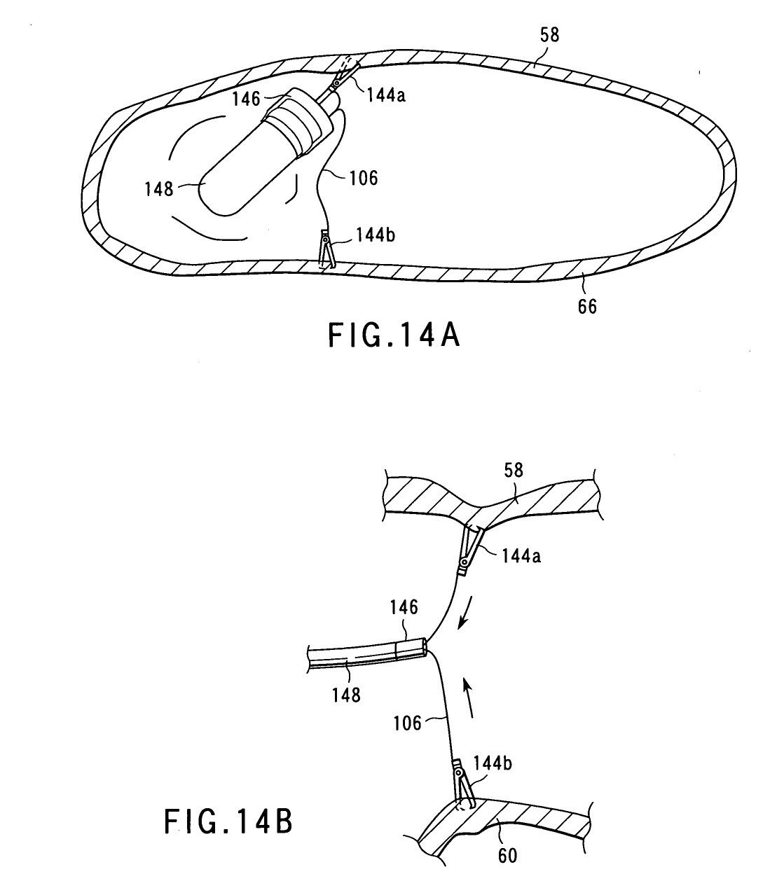

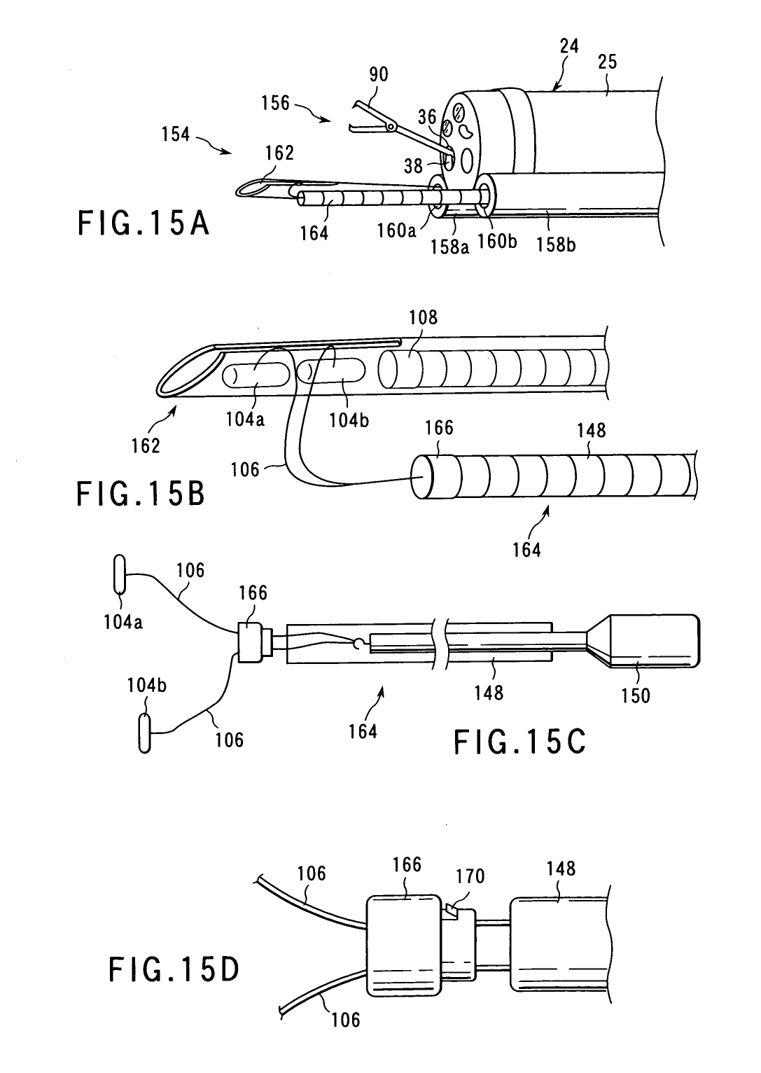

Next, a treatment method of a third embodiment of the present invention will be described with reference to FIGS. 13A to 14B. The treatment method of this embodiment has a formation of the false esophagus 18, which is different from the formation of the false esophagus 18 in the first embodiment. An endoscope apparatus 128 of this embodiment to form the false esophagus 18 will be described with reference to FIGS. 13A to 13C. The endoscope apparatus 128 is different from the endoscope apparatus 22 (FIG. 3A) of the first embodiment forming the false esophagus, with respect to the only structure explained below.

The bending portion 70 operated to bend (FIG. 5A) is provided at the insertion portion 25 of the endoscope 24 of the endoscope apparatus 128. The endoscope 24 is fitted in an inner cavity of a distal tube 130 as shown in FIG. 13A. Furthermore, a forceps aperture 134 into which a retaining forceps 132 is to be inserted is bored through the distal tube 130, in a direction of the central axis of the distal hood. A sheath 136 is connected to an opening portion of a rear end side of the forceps aperture 134. A cross section of the inner cavity of the sheath 136 perpendicular in the lengthwise direction has substantially the same shape as a cross section of the forceps aperture 134 perpendicular in the lengthwise direction. The retaining forceps 132 is inserted into the inner cavity of the sheath 136 and the forceps aperture 134. In other words, a forceps channel 138 is formed by the inner cavity of the sheath 136 and the forceps aperture 134. The retaining forceps 132 has a first clip applicator portion 140a, a second clip applicator portion 140b, and a binding forceps portion 142.

The first and second clip applicator portions 140a and 140b have the same structure as the clip applicator 34 (FIG. 3A) of the first embodiment. The first and second clip applicator portions 140a and 140b have first and second clip portions 144a and 144b, respectively, which are the same as the clip member 40 (FIG. 3A) of the first embodiment. The first clip portion 144a and the second clip portion 144b are connected to each other by a thread 106 as first and second thread elements as shown in FIG. 13B.

On the other hand, a cylindrical fastening member 146 is provided on a distal side of the binding forceps portion 142. The fastening member 146 is formed of an elastic member such as a silicon tube or the like. A cylindrical applicator 148 capable of pushing the fastening member 146 is provided on a rear end side of the binding forceps portion 142. A thread pulling handle 150 is contained in the applicator 148. The handle 150 is capable of freely advancing or retreating relative to the applicator 148.

The thread 106 connecting the first clip portion 144a and the second clip portion 144b is folded in the middle and doubled. The doubled portion of the thread 106 is inserted into the fastening member 146 and the applicator 148. The fastening member 146 binds and fastens the doubled portion of the thread 106. The folded portion of the thread 106 is engaged on a hook of a distal portion of the handle 150. When the applicator 148 is pushed relative to the handle 150, the fastening member 146 is pushed by the applicator 148 and the thread 106 between the fastening member 146 and the first and second clip portions 144a, 144b is thereby shortened.

The forceps aperture 134 has one side portion 134a and other side potion 134b into which the first and second clip applicator portions 140a, 140b are inserted, and a central portion 134c into which the binding forceps portion 142 is inserted, as shown in FIG. 13C. Coupling portions are provided between the side portions 134a and 134b and the central portion 134c such that the thread 106 is inserted into the coupling portions. The inner cavity of the sheath 136 has the same shape.

Formation of the false esophagus 18 according to this embodiment will be described with reference to FIGS. 14A and 14B. First, the insertion portion 25 is perorally inserted into the stomach. The insertion portion 25 is operated to advance and retreat, rotate or bend, and the first clip portion 144a is thereby directed to a region of interest. After that, the first clip applicator portion 140a is operated such that the first clip portion 144a is pushed toward the region of interest, that the first and second grasping portions grasp the region of interest and that the first and second prongs 44a and 44b stick into the anterior wall of stomach 58. As a result, the first clip portion 144a is engaged and retained in the region of the inner surface of the anterior wall of stomach 58 in which it needs to be joined as shown in FIG. 14A. Similarly, the second clip portion 144b is engaged and retained in the region of the inner surface of the posterior wall of stomach in which it needs to be joined.

The applicator 148 is pushed relative to the handle 150 after the first clip portion 144a and the second clip portion 144b are retained. As a result, the fastening member 146 is pushed by the applicator 148 to fasten the thread 106 as represented by an arrow in FIG. 14B. Thus, the distance between the first clip portion 144a and the second clip portion 144b is shortened, the anterior wall of stomach 58 and the posterior wall of stomach 60 are pulled to each other, and a portion of the inner surface of the anterior wall of stomach 58 and that of the posterior wall of stomach 60 are joined. After that, the engagement between the handle 150 and the thread 106 is released, the applicator 148 and the handle 150 are pulled into the forceps aperture 134, and the first clip portion 144a and the second clip portion 144b and the thread 106 are retained. The joining condition of the anterior wall of stomach 58 and the posterior wall of stomach 60 is maintained by the elastic force of the fastening member 146. After that, the first clip portion 144a and the second clip portion 144b and the thread 106 are retained at some positions spaced in a direction parallel to the axis of the esophagus, from the fundus of stomach 20 side of the gastroesophageal boundary 14 to the anal side. The false esophagus 18 is thereby formed.

According to the endoscope apparatus 128 of this embodiment forming the false esophagus 18, the forceps channel 138 is formed by the distal hood and the sheath 136. For this reason, the endoscope 24 including a channel having a great diameter does not need to be utilized. Therefore, an endoscope having a small outer diameter can be employed as the endoscope 24.

In addition, the first clip portion 144a is engaged with the inner surface of the anterior wall of stomach 58, the second clip portion 144b is engaged with the inner surface of the posterior wall of stomach 60, and the thread 106 is fastened. Thus, the distance between the first clip portion 144a and the second clip portion 144b is shortened and a portion of the inner surface of the anterior wall of stomach 58 and that of the posterior wall of stomach 60 are joined. In other words, a portion of the inner surface of the anterior wall of stomach 58 and that of the posterior wall of stomach 60 can be joined while the stomach is expanded. For this reason, a field of view can be ensured sufficiently during the joining operation, and the joining operation can be executed easily and certainly.

The distance between the first clip portion 144a and the second clip portion 144b is maintained by fastening the thread 106 and then the joining between a portion of the inner surface of the anterior wall of stomach 58 and that of the posterior wall of stomach 60 is kept. For this reason, the joining condition can be released by cutting the thread 106 between the fastening member 146 and the first and second clip portions 144a, 144b. Therefore, the stomach can easily be returned to its initial shape.

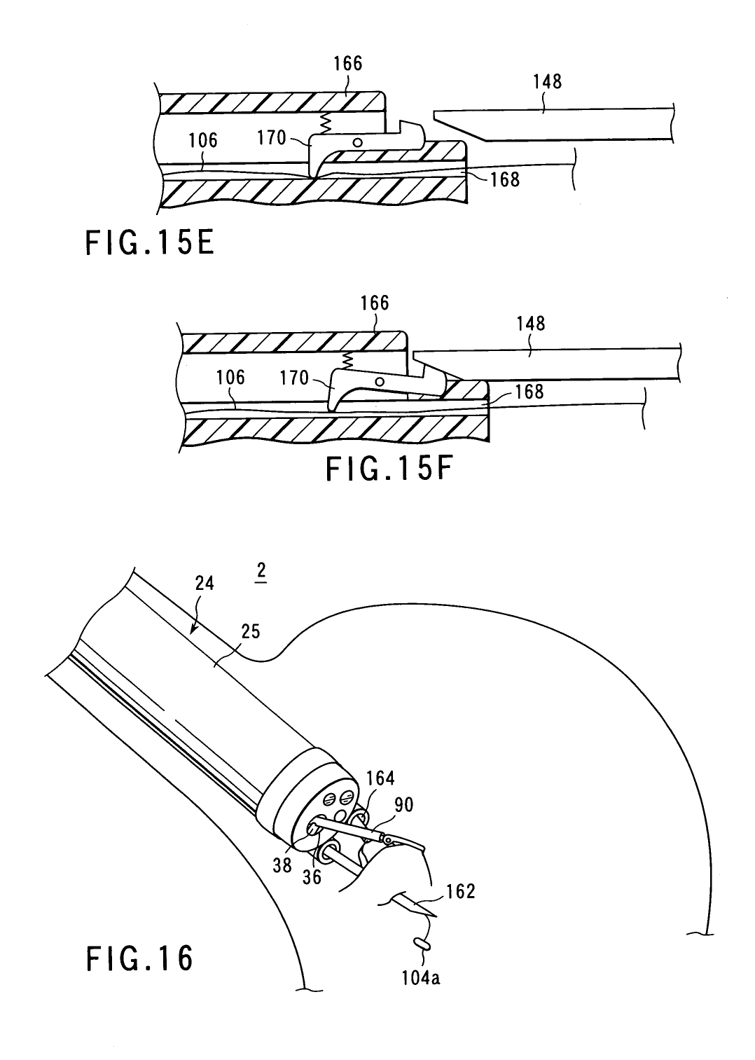

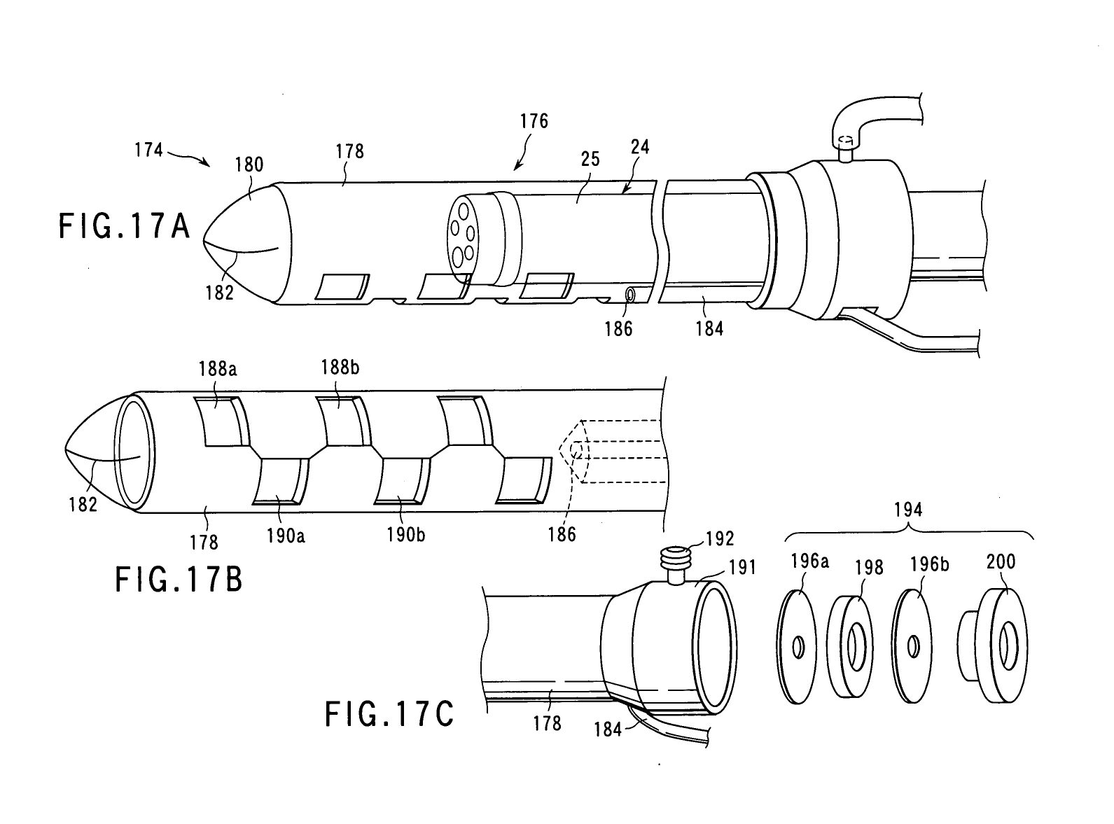

Next, a treatment method of a fourth embodiment of the present invention will be described with reference to FIGS. 15A to 16. The treatment method of this embodiment has a formation of the false esophagus 18, which is different from the formation of the false esophagus 18 in the first embodiment. An endoscope apparatus 154 of this embodiment to form the false esophagus 18 will be described with reference to FIGS. 15A to 15F.

As shown in FIG. 15, the endoscope apparatus 154 has the endoscope 24, the first forceps channel 36 and the elevator 38, which are similar to the endoscope 24, the forceps channel 36 and the elevator 38 (FIG. 3A) of the endoscope apparatus 22 of the first embodiment for forming the false esophagus 18. The grasping forceps 90, which is similar to the grasping forceps 90 (FIG. 3A) of the endoscope apparatus 22 of the first embodiment, is inserted into the first forceps channel 36.

On the other hand, two sheaths 158a and 158b are attached to an outer surface of the endoscope 24 and arranged side by side. A second forceps channel 160a and a third forceps channel 160b are formed by inner cavities of the sheathes 158a and 158b. A puncturing needle 162 is inserted into the second forceps channel 160a and a binding forceps 164 is inserted into the third forceps channel 160b.

The puncturing needle 162 is substantially the same as the puncturing needle 100 of the endoscope apparatus 22 of the first embodiment as shown in FIG. 15B. However, a slit is formed at a distal portion on an outer peripheral wall of the puncturing needle 162, in the axial direction of the puncturing needle 162. The thread 106 extends through the slit. On the other hand, the binding forceps 164 is substantially the same as the binding forceps portion 142 (FIG. 13B) of the endoscope apparatus 128 according to the third embodiment, but is different therefrom in view of a fastening member 166. A detailed structure of the fastening member 166 will be explained below.

The fastening member 166 fastens the thread 106 extending from the first and second T-bars 104a and 104b as shown in FIG. 15C. A small-diameter portion which is to be fitted in the inner cavity of the applicator 148 is provided on a rear side of the fastening member 166. Two insertion apertures 168 (FIGS. 15E and 15F) in which the threads 106 are inserted are formed to the fastening member 166, parallel to a direction a central axis thereof. The threads 106 extending from the first T-bar 104a and the second T-bar 104b are inserted into the insertion apertures 168, respectively.

An urging member 170 limiting the advance and retreat of the thread 106 is provided in the vicinity of the insertion apertures 168 of the fastening member 166 as shown in FIG. 15E. The urging member 170 extends in the direction of the central axis of the fastening member 166. A central portion of the urging member 170 is supported by the fastening member 166 such that the urging member 170 rotate about its central potion and a distal end portion thereof is urged into the insertion apertures 168 by an elastic member and a proximal end portion thereof project from an outer peripheral wall of the small-diameter portion. If the fastening member 166 is not mounted on the applicator 148, the distal end portion of the urging member 170 limits the advance and retreat of the threads 106 by urging the threads 106 in the insertion apertures 168. On the other hand, if the fastening member 166 is mounted on the applicator 148, the proximal end portion of the urging member 170 is pressurized inwardly by an inner peripheral surface of the applicator 148 and the distal end portion of the urging member 170 releases urging of the threads 106 in the insertion apertures 168 as shown in FIG. 15F. In this state, the threads 106 can freely advance or retreat relative to the fastening member 166.

Formation of the false esophagus 18 according to the fourth embodiment will be described with reference to FIG. 16. First, the insertion portion 25 is perorally inserted into the stomach. The grasping forceps 90 projects from the first forceps channel 36, and the portion of the inner surface of the anterior wall of stomach to be joined is grasped by the grasping forceps 90. This grasping operation is the same as that in the first embodiment.

After that, the grasping forceps 90 is moved in a direction of going away from the anterior wall of stomach by the elevator 38 and a part of the anterior wall of stomach is raised. At this time, since the grasping forceps 90 sufficiently grasps the anterior wall of stomach, even the muscularis is certainly raised. The puncturing needle 162 is made to stick into a proximal portion of the raised part of the anterior wall of stomach, the first T-bar 104a is discharged from the puncturing needle 162, and the puncturing needle 162 is extracted from the anterior wall of stomach. The sticking, releasing ands extracting operations are the same as those of the first embodiment. As a result, the first T-bar 104a is engaged in the portion of the inner surface of the anterior wall of stomach to be joined and the thread 106 is inserted through the muscularis of the anterior wall of stomach. Furthermore, the second T-bar 104b is engaged with the portion of the inner surface of the posterior wall of stomach to be joined, which corresponds to the portion of the inner surface of the anterior wall of stomach to be joined.

After that, the fastening member 166 is pushed integrally with the applicator 148 relative to the threads 106 to fasten the threads 106. As a result, the distance between the first T-bar 104a and the second T-bar 104b is shortened, the anterior wall and the posterior wall are pulled up to each other, and a portion of the inner surface of the anterior wall of stomach and a portion of the inner surface of the posterior wall of stomach are joined. The handle 150 is pushed to the distal side relative to the applicator 148 and the fastening member 166 is discharged from the applicator 148. As a result, the urging member 170 limits the advancing and retreating of the threads 106 in the insertion apertures 168 by the elastic force of the elastic member. The engagement of the handle 150 and the threads 106 is released. The applicator 148 and the handle 150 are pulled into the forceps aperture. The retained members are retained. The advancing and retreating of the threads 106 are limited by the function of the urging member 170. The joined state of the anterior wall and the posterior wall is maintained. After that, false esophagus 18 is formed by retaining the first T-bar 104a, the second T-bar 104b, and the threads 106 at some positions spaced in a direction parallel to the axis of the esophagus, from the fundus of stomach 20 side of the gastroesophageal boundary 14 to the anal side, similarly to the first embodiment.

In this embodiment, the portions of the gastric walls to be joined are raised by the grasping forceps 90. The puncturing needle 162 is made to stick into the proximal parts of the raised portions. The first T-bar 104a is discharged from the puncturing needle 162. The puncturing needle 162 is extracted from the gastric walls. At this time, since the grasping forceps 90 sufficiently grasps the gastric walls, the threads 106 are inserted through the muscularis. For this reason, the first T-bar 104a, the second T-bar 104b, and the threads 106 are sufficiently fixed on the gastric walls and are rarely detached therefrom. Therefore, the anterior wall and the posterior wall can be certainly pulled to each other.

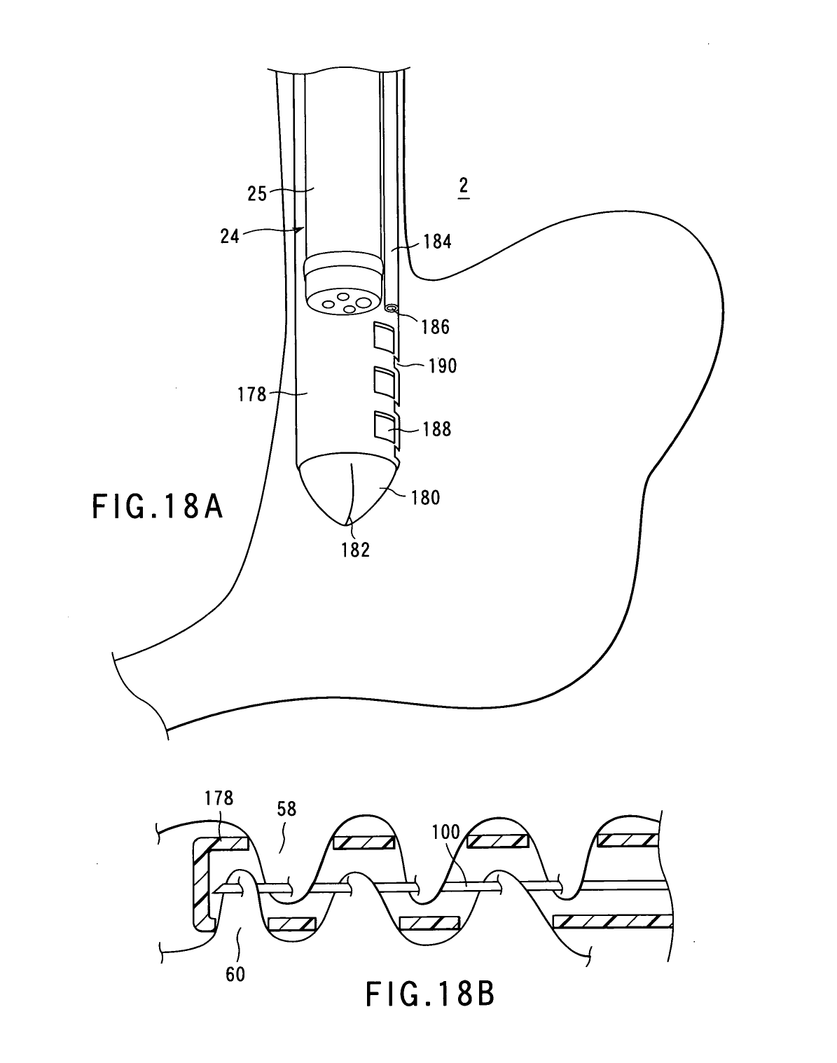

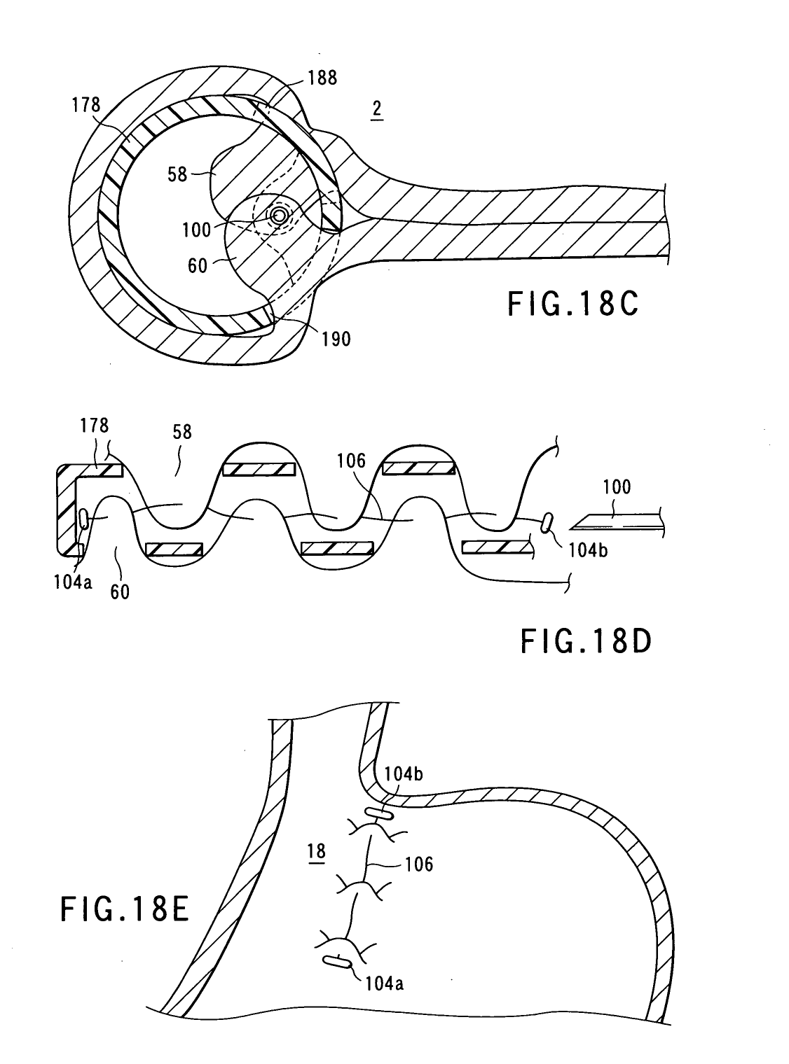

Next, a treatment method of a fifth embodiment of the present invention will be described with reference to FIGS. 17A to 18E. The treatment method of this embodiment has a formation of the false esophagus 18, which is different from the formation of the false esophagus 18 in the first embodiment. An endoscope apparatus 174 of this embodiment to form the false esophagus 18 will be described with reference to FIGS. 17A to 17C.

As shown in FIG. 17A, the endoscope apparatus 174 has an over tube 178 as a tube-like member through which the insertion portion 25 of the endoscope 24 is inserted. A hood portion 180 is provided at a distal portion of the overtube 178. The hood portion 180 is provided to close the opening at the distal portion of the overtube 178, and has a tapered shape so as to easily enter a patient's mouth. A plurality of slits 182 extending from the top to the foot of the hood portion 180 are formed on the hood portion 180. If the insertion portion 25 is pushed inside the overtube 178, the distal portion of the insertion portion 25 pushes and expands the hood portion 180 and projects from the overtube 178. The slits 182 are just closed without clearance during a suction operation inside the overtube 178 to be described later.

A sheath 184 is provided on an inner surface of the overtube 178, in an axial direction thereof. The sheath 184 extends from a connecting portion 190 provided at a proximal portion of the overtube 178. An overtube channel 186 is formed by the sheath 184. The overtube channel 186 is available for irrigation, insufflation or suction. A puncturing needle 100 having the same structure as that of the puncturing needle 100 (FIG. 6C) of the endoscope apparatus 72 (FIG. 6A) of the first embodiment is inserted into the overtube channel 186. Suction inside overtube 178 can also be executed by the channel formed on the endoscope 24.

A plurality of side apertures 188, 190 are formed to an outer peripheral wall of the overtube 178. The side apertures 188, 190 include first side aperture group 188a, . . . , 188n facing the anterior wall of stomach and second side aperture group 190a, . . . , 190n facing the posterior wall of stomach, as shown in FIG. 17B. The first side apertures 188a, . . . , 188n are spaced from each other in the direction of the central axis of the overtube 178. The second side apertures 190a, . . . , 190n are also arranged similarly. Moreover, the first side apertures 188a, . . . , 188n and the second side apertures 190a, . . . , 190n are arranged parallel. The first side apertures 188a, . . . , 188n and the second side apertures 190a, . . . , 190n are offset in the direction of the central axis of the overtube 178. Furthermore, slits are formed between the first side apertures 188 and the second side apertures 190 arranged adjacently.

A distal portion of the overtube channel 186 is arranged on a rear side of the first side apertures 188 and the second side apertures 190. The overtube channel 186 is arranged in the middle of the first side aperture group 188a, . . . , 188n and the second side aperture group 190a, 1 . . . , 90n, in a circumferential direction about the central axis of the overtube 178.

A suction connector 192 is provided at the connecting portion 190 of the proximal portion of the overtube 178 as shown in FIG. 17C. An inner end portion of the suction connector 192 communicates with the interior of the overtube 178. An outer end portion of the suction connector 192 can be connected to an external pump. A valve mechanism 194 inserting the endoscope 24 airtightly is provided at the connecting portion 190. The valve mechanism 194 has two sheet-like airtight valves 196a and 196b which are formed of elastic members. A ring-shaped member 198 is arranged between the airtight valves 196a and 196b. The airtight valves 196a and 196b and the ring-shaped member 198 are screwed in the connecting portion 190 by a pressing member 200. The ring-shaped member 198 and the pressing member 200 have a central aperture whose diameter is greater than an outer diameter of the endoscope 24. The airtight valves 196a and 196b have a central aperture whose diameter is smaller than the outer diameter of the endoscope 24.

Formation of the false esophagus 18 according to this embodiment will be described with reference to FIGS. 18A to 18E. First, the overtube 178 in which the insertion portion 25 is inserted is perorally inserted into the stomach as shown in FIG. 18A. At this time, the field of view of the endoscope 24 can be ensured by making the insertion portion 25 project from the distal portion of the overtube 178.

Subsequently, the overtube 178 is arranged at a position where the false esophagus 18 is to be formed. Furthermore, the overtube 178 is rotated about its central axis. Thus, the first side aperture group 188a, . . . , 188n is made to face the anterior wall of stomach, and the second side aperture group 190a, . . . , 190n is made to face the posterior wall of stomach. After that, the interior of the overtube 178 is sucked through the suction connector 192 of the connecting portion 190. As a result, the anterior wall is pulled in the interior of the overtube 178 through the first side aperture group 188a, . . . , 188n and the posterior wall is pulled in the interior of the overtube 178 through the second side aperture group 190a, . . . , 190n.

In this state, the needle 100 projects from the overtube channel 186 under observation of the endoscope 24. The puncturing needle 100 alternately sticks in and passes through the pulled portions of the anterior wall of stomach 58 and the pulled portions of the posterior wall of stomach 60, from the proximal side, as shown in FIG. 18B. The anterior wall of stomach 58 and the posterior wall of stomach 60 are mutually stacked in the lengthwise direction of the overtube 178 as shown in FIG. 18C.

If it is confirmed that the puncturing needle 100 passes through the gastric wall which is closest to the anal side, the first T-bar 104a is discharged from the needle 100. These sticking and discharging operations are the same as those of the first embodiment. After that, the puncturing needle 100 is completely extracted from the pulled portions of the anterior wall of stomach 58 and the pulled portions of the posterior wall of stomach 60. The thread 106 is made to pass through the pulled portions of the anterior wall of stomach 58 and the pulled portions of the posterior wall of stomach 60, sequentially. The second T-bar 104b is discharged as shown in FIG. 18D.

After that, the overtube 178 is operated such that the gastric walls are extracted from the overtube 178 through the first and second side apertures 188 and 190 and that the thread 106 is extracted through the slits between the first side apertures 188 and the second side apertures 190.

As a result, the thread 106 passes through the anterior wall and the posterior wall, alternately, and is fixed by the first and second T-bars 104a and 104b which are engaged with the gastric walls, as shown in FIG. 18E. Thus, the false esophagus 18 is formed.

In this embodiment, a plurality of portions of the anterior wall and the posterior wall which are to be joined are pulled into the overtube 178 by one-time suction. The plural portions can be joined by one-time sticking operation. For this reason, the false esophagus 18 can be formed more easily and quickly.

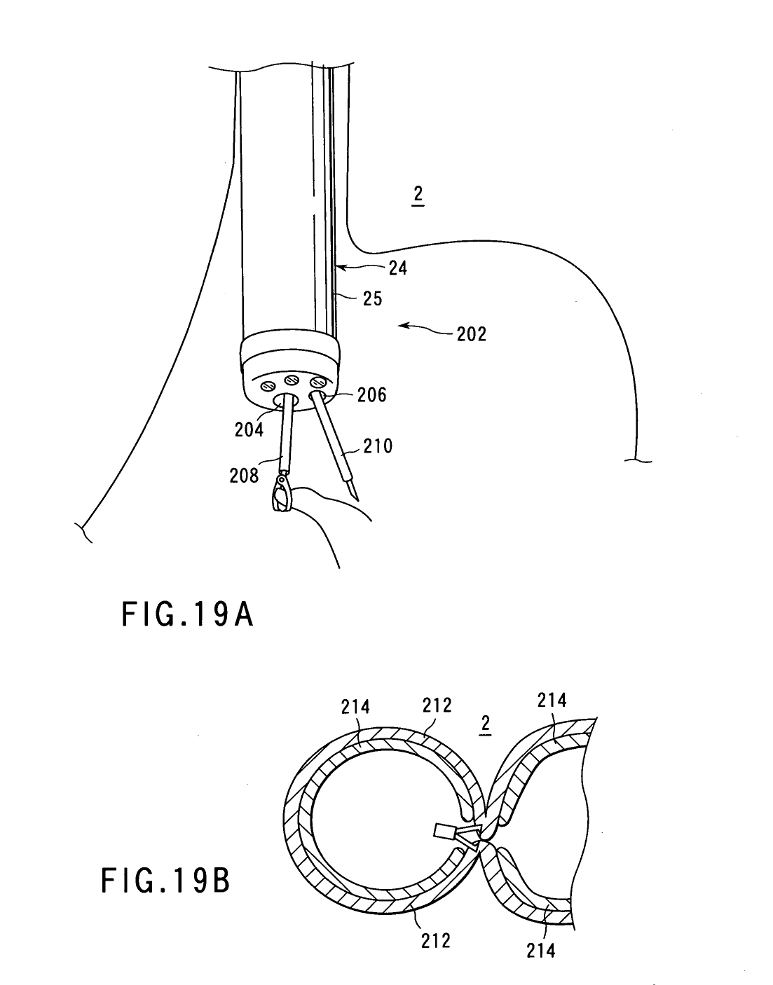

Next, a treatment method according to a sixth embodiment of the present invention will be described with reference to FIGS. 19A and 19B. The treatment method further comprises a resecting operation besides the formation of the false esophagus 18 according to the first embodiment. The resecting operation to be explained below can be added to any of the above-described embodiments. Since the resecting operation can be executed by a general method of demucosation, for example, a method of EMR, its detailed description is omitted here.

An endoscope apparatus 202 for the resecting operation will be explained with reference to FIG. 19A. A first forceps channel 204 and a second forceps channel 206 are provided at the insertion portion 25 of the endoscope 24 of the endoscope apparatus 202. An elevator (not shown) is provided on each of the first forceps channel 204 and the second forceps channel 206. A grasping forceps 208 capable of grasping a living tissues is inserted into the first forceps channel 204. A high-frequency knife 210 capable of cutting the living tissues is inserted into the second forceps channel 206.

The resecting operation is explained with reference to FIGS. 19A and 19B. First, the insertion portion 25 is perorally inserted into the stomach as shown in FIG. 19A. The grasping forceps 208 is made to project from the first forceps channel 204 of the insertion portion 25. The portions of the gastric walls to be joined are grasped by the grasping forceps 208. The grasping forceps 208 is moved in a direction of going away from the gastric walls by the elevator and the gastric walls are thereby raised. Subsequently, the high-frequency knife 210 is made to project from the second forceps channel 206. A lower side of the raised gastric walls is cut by the high-frequency knife 210 and mucous membranes are resected. The portions from which the mucous membranes 214 are resected are mutually joined as shown in FIG. 19B. In other words, muscularis 212 of the anterior wall of stomach and muscularis 212 of the posterior wall of stomach are mutually joined directly.

According to this embodiment, the mucous membranes 214 of the walls of stomach to be joined are resected, and the muscularis 212 of the walls of stomach are directly joined. For this reason, adhesion occurs at the joined portions and the walls of stomach can be thereby joined more firmly.

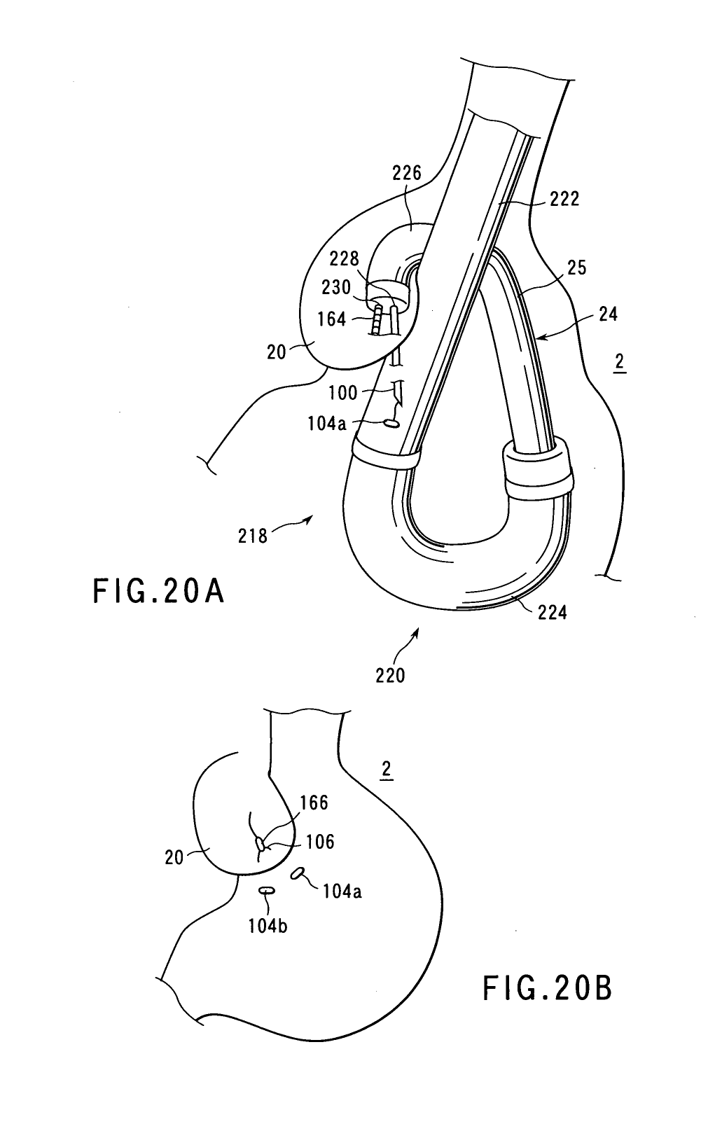

Next, a treatment method according to a seventh embodiment of the present invention will be described with reference to FIGS. 20A and 20B. This embodiment has winding the fundus of stomach, which is different from the winding of the first embodiment, and fixing the fundus of stomach, which is different from the fixing of the first embodiment. An endoscope apparatus 218 of this embodiment for the winding and fixing operations will be described with reference to FIG. 20A.

The endoscope apparatus 218 has an overtube 222 as a tube-like member through which the insertion portion 25 of the endoscope 24 is inserted. The overtube 222 has a first bending portion 224 which is operated to bend integrally with the inserted endoscope 24. The endoscope 24 has a second bending portion 226 which is operated to bend. A first forceps channel 228 and a second forceps channel 230 are formed to the insertion portion 25 of the endoscope 24. The puncturing needle 100 having the same structure as that of the puncturing needle 100 (FIG. 15B) of the endoscope apparatus 154 (FIG. 15A) of the fourth embodiment forming the false esophagus 18 is inserted into the first forceps channel 228. The binding forceps 164 having the same structure as that of the binding forceps 164 of the endoscope apparatus 154 (FIG. 15A) of the fourth embodiment, is inserted into the second forceps channel 230.

The winding and fixing operations according to this embodiment will be described with reference to FIGS. 20A and 20B. The insertion portion 25 of the endoscope 24 is inserted into the overtube 222 such that the distal portion of the insertion portion 25 projects from the overtube 222. The insertion portion 25 and the overtube 222 are perorally inserted into the stomach. The first bending portion 224 of the overtube 222 is bent such that the distal portion of the insertion portion 25 is directed to the fundus of stomach 20. The first bending portion 224 is kept bent and the insertion portion 25 is pushed relative to the overtube 222. As a result, the distal portion of the insertion portion 25 is engaged with the fundus of stomach 20.

The insertion portion 25 is further pushed relative to the overtube 222. The fundus of stomach 20 engaged with the distal portion of the insertion portion 25 is moved beyond the false esophagus 18 inside the peritoneal cavity. After that, the second bending portion 226 of the insertion portion 25 is bent such that the insertion portion 25 is wound around the false esophagus 18 from the peritoneal cavity side. As a result, the fundus of stomach 20 is wound around the false esophagus 18 from the peritoneal cavity side. The second bending portion 226 of the insertion portion 25 is sufficiently bent and a portion of the fundus of stomach 20 on the peritoneal cavity 2 side is made to contact a portion of the false esophagus 18 on the peritoneal cavity 2 side.

In this state, the puncturing needle 100 projects from the first forceps channel 228, passes through the body wall of the fundus of stomach 20, projects into the peritoneal cavity, passes through the body wall of the false esophagus 18, and projects again into the peritoneal cavity. The first T-bar 104a is discharged from the puncturing needle 100 and engaged with the body wall of the false esophagus 18, similarly to the fourth embodiment, as shown in FIG. 20A. After that, the puncturing needle 100 is extracted from the body wall portion of the false esophagus 18 and the body wall of the fundus of stomach 20, to return to the interior of the fundus of stomach 20. The second T-bar 104b is engaged with the body wall portion of the false esophagus 18, similarly to the first T-bar 104a. After that, the thread 106 is fastened by the fastening member, similarly to the fourth embodiment. The distance between the first and second T-bars 104a, 104b and the fastening member is shortened. The first T-bar 104a, the second T-bar 104b, and the fastening member are maintained in this state. Thus, the fundus of stomach 20 is wound around the false esophagus 18 and maintained in this wound state.

In this embodiment, as the distal portion of the insertion portion 25 is engaged with the fundus of stomach 20, the field of view of the endoscope 24 may be blocked. For this reason, to appropriately execute the winding and fixing operations, the following device and method may be applied.

To ensure the field of view of the endoscope 24, a tube-shaped distal hood may be provided at the distal portion of the insertion portion 25. In addition, to confirm the current condition of the winding operation, the winding may be executed while looking through a fluoroscope. The current condition of the winding operation may be confirmed by surgically inserting a rigidscope into the peritoneal cavity though.

In addition, a device detecting the deformed state of the insertion portion 25 may be employed. The following device is known as such as a detecting device. This detecting device has a plurality of coils built in the insertion portion 25. The coils are spaced in the direction of the central axis of the insertion portion 25 and make outputs in accordance with an applied magnetic field. The positions of the coils to the source of the magnetic field are calculated based on the outputs and the deformed condition of the insertion portion 25 can be confirmed from the positions of the coils. This deformed condition can be displayed on a monitor.

In this embodiment, the distal portion of the insertion portion 25 is engaged with the fundus of stomach 20. The insertion position 25 is pushed relative to the overtube 222. The second bending portion 226 of the insertion portion 25 is bent to wind around the false esophagus 18. As the fundus of stomach 20 is thus wound around the false esophagus 18 from the peritoneal cavity side, the fundus of stomach 20 can be sufficiently wound around the false esophagus 18.

In the above-described first to seventh embodiments, the fundus of stomach is wound around the false esophagus from the peritoneal cavity side. In the first to sixth embodiments, the fundus of stomach is fixed to the false esophagus at some positions, on the peritoneal cavity side of the false esophagus. However, the fundus of stomach may not be wound around the false esophagus, but may be fixed to the false esophagus at only one position, on the peritoneal cavity side of the false esophagus. As the false esophagus exists in the peritoneal cavity and is surrounded by various organs, the false esophagus is sandwiched between the fundus of stomach and the other organs due to the expansion of the fundus of stomach, and is thereby pressed and enclosed. Thus, the reflux is prevented.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A treatment method comprising:
perorally,
forming a false esophagus in a stomach by pulling an anterior wall and a posterior wall of stomach to each other and by joining a part of an inner surface of the anterior wall and a part of an inner surface of the posterior wall to each other to extend an esophagus;
making a part of a peritoneal cavity side of a fundus of stomach approach a part of a peritoneal cavity side of the false esophagus; and
fixing the part of the fundus of stomach and the part of the false esophagus which are made to approach to each other.

2. The treatment method according to claim 1, wherein the forming includes:
perorally inserting a tube-like member into the stomach;
covering one side of an outer peripheral surface of the tube-like member with the anterior wall of stomach and the other side thereof with the posterior wall of stomach;
directing a communication portion formed on the outer peripheral surface of the tube-like member to a part to be joined; and
making joining member project from an interior of the tube-like member through the communication portion.

3. The treatment method according to claim 2, wherein the covering includes sucking an interior of the tube-like member.

4. The treatment method according to claim 1, wherein the forming includes:
perorally inserting a retained member into the stomach;
engaging a first engaging portion of the retained member with any one of the anterior wall of and the posterior wall of stomach;
moving the retained member toward the other one of the anterior wall and the posterior wall with maintaining the engagement of the first engaging portion with any one of the anterior wall and the posterior wall;
engaging a second engaging portion of the retained member with the other one of the anterior wall and the posterior wall; and
retaining the retained member with maintaining the engagement of the first engaging portion with any one of the anterior wall and the posterior wall and maintaining the engagement of the second engaging portion with the other one of the anterior wall and the posterior wall.

5. The treatment method according to claim 1, wherein the forming includes:
perorally inserting a retained member into the stomach;
engaging a first engaging portion of the retained member with any one of the anterior wall and the posterior wall;
engaging a second engaging portion of the retained member with the other one of the anterior wall and the posterior wall;
shortening a distance between the first engaging portion and the second engaging portion with maintaining the engagement of the first engaging portion with any one of the anterior wall and the posterior wall and with maintaining the engagement of the second engaging portion with the other one of the anterior wall and the posterior wall; and
retaining the retained member with maintaining the engagement of the first engaging portion with any one of the anterior wall and the posterior wall, with maintaining the engagement of the second engaging portion with the other one of the anterior wall and the posterior wall, and with maintaining a state of shortening the distance between the first engaging portion and the second engaging portion.

6. The treatment method according to claim 1, wherein the forming includes:
perorally inserting a tube-like member into the stomach;
pulling the anterior wall of stomach into the tube-like member through the first communication portion formed to the tube-like member;
pulling the posterior wall of stomach into the tube-like member through a second communication portion formed to the tube-like member; and
joining the anterior wall and the posterior wall pulled into the tube-like member to each other inside the tube-like member.

7. The treatment method according to claim 6, wherein the pulling of the anterior wall of stomach or the pulling of the posterior wall of stomach includes the sucking of the interior of the tube-like member.

8. The treatment method according to claim 1, wherein the forming includes resecting at least one area of mucous membrane on a part of the inner surfaces joined to each other before the joining.

9. The treatment method according to claim 1, wherein making the part of the fundus of stomach approach the part of the false esophagus includes:
perorally inserting an endoscope into the stomach;
bending the endoscope and engaging a distal portion of the endoscope with the fundus of stomach; and
further bending the bent portion of the endoscope with maintaining the engagement of the distal portion of the endoscope with the fundus of stomach.

10. The treatment method according to claim 1, wherein the fixing of the part of the fundus of stomach and the part of the false esophagus includes:
perorally inserting a tube-like member into the stomach;
pulling a part of a body wall of the fundus of stomach and a part of a body wall of the false esophagus into the tube-like member through a communication portion formed to the tube-like member with the part of the body wall of the fundus of stomach and the part of the body wall of the false esophagus being stacked; and
fixing the part of the body wall of the fundus of stomach and the part of the body wall of the false esophagus, which are pulled into the tube-like member, to each other.

11. The treatment method according to claim 1, wherein making the part of the fundus of stomach approach the part of the false esophagus includes winding the fundus of stomach around the peritoneal cavity side of the false esophagus.

12. The treatment method according to claim 11, wherein the winding includes:
perorally inserting an endoscope apparatus into the stomach;
bending a bending portion of the endoscope apparatus and engaging a distal portion of the endoscope apparatus with the fundus of stomach;
further bending the bending portion of the endoscope apparatus with maintaining the engagement of the distal portion of the endoscope apparatus with the fundus of stomach; and
rotating a proximal end side of the endoscope apparatus about an central axis of the endoscope apparatus to make the distal portion of the endoscope apparatus rotate in a peripheral direction of the false esophagus.

13. The treatment method according to claim 11, wherein the fixing includes fixing the fundus of stomach at a plurality of positions spaced in the peripheral direction of the false esophagus, on the peritoneal cavity side of the false esophagus.

14. The treatment method according to claim 11, wherein the winding includes:
perorally inserting a tube-like member capable of bending operation and an endoscope which is inserted into the tube-like member and whose distal portion projects from a distal portion of the tube-like member, into the stomach;
bending the tube-like member and engaging the distal portion of the endoscope with the fundus of stomach;
pushing the endoscope relative to the tube-like member with maintaining the engagement of the distal portion of the endoscope with the fundus of stomach; and
bending a bending portion of the endoscope to wind the endoscope around the false esophagus from the peritoneal cavity side.

15. An endoscope apparatus configured to be perorally inserted into a stomach to form a false esophagus in the stomach by pulling an anterior wall of stomach and a posterior wall of stomach to each other and joining a part of an inner surface of the anterior wall and a part of an inner surface of the posterior wall to each other so as to extend an esophagus.

16. An endoscope apparatus comprising:
an insertion portion perorally inserted into a stomach;
an observing optical system provided in the insertion portion for observation of a body cavity;
a tube-like member provided integrally or separately at a distal portion of the insertion portion;
a first channel and a second channel which are formed at the insertion portion or provided at the insertion portion so as to open to an inner side of the tube-like member;
a communication portion formed to an outer peripheral wall of the tube-like member to make the interior of the tube-like member communicate with an exterior thereof;
a retained member having a first engaging portion and a second engaging portion which are engaged on a body wall; and
a retaining forceps having the retained member at a distal portion thereof, being inserted into the first channel or the second channel, projecting the retained member from the interior of the tube-like member to the exterior thereof through the communication portion, engaging one of the first and second engaging portions on one of a anterior wall of stomach and a posterior wall of stomach, engaging the other of the first and second engaging portions on the other of the anterior wall and the posterior wall, and retaining the retained member with the first or second engaging portions being engaged on the anterior wall or the posterior wall.

17. The endoscope apparatus according to claim 16, further comprises an adjusting member which adjusts a direction of advance of the retaining forceps provided at a portion close to an opening of at least one of the first channel and the second channel in which the retaining forceps is inserted.

18. The endoscope apparatus according to claim 17, wherein the adjusting member includes an elevator.

19. The endoscope apparatus according to claim 16, wherein the retaining forceps includes a bending portion which is bent to move the retained member.

20. The endoscope apparatus according to claim 16, wherein:
the retained member includes a clip member having a first grasping portion and a second grasping portion which are relatively opened or closed, the first engaging portion includes a first prong provided on an inner side surface of a distal portion of the first grasping portion, and the second engaging portion includes a second prong provided on an inner side surface of a distal portion of the second grasping portion; and
the retaining forceps includes a clip applicator opening or closing, moving, and retaining the clip member.

21. An endoscope apparatus comprising:
an insertion portion perorally inserted into a stomach;
an observing optical system provided in the insertion portion for observation of a body cavity;
a channel formed at the insertion portion or provided at the insertion portion;
a retained member having a first engaging portion and a second engaging portion which are engaged on a body wall; and
a retaining forceps having the retained member at a distal portion thereof, being inserted into the channel, engaging one of the first and second engaging portions with one of the anterior wall and the posterior wall, moving the retained member with one of the first and second engaging portions being engaged with one of the anterior wall and the posterior wall, engaging the other one of the first and second engaging portions with the other one of the anterior wall and the posterior wall, and retaining the retained member with the first or second engaging portions being engaged on the anterior wall or the posterior wall.

22. The endoscope apparatus according to claim 21, wherein:
the retained member includes a clip member having a first grasping portion and a second grasping portion which are relatively opened or closed, the first engaging portion includes a first prong provided on an inner side surface of a distal portion of the first grasping portion, and the second engaging portion includes a second prong provided on an inner side surface of a distal portion of the second grasping portion; and
the retaining forceps includes a clip applicator opening or closing, moving, and retaining the clip member.

23. An endoscope apparatus comprising:
an insertion portion perorally inserted into a stomach;
an observing optical system provided in the insertion portion for observation of a body cavity;
a channel formed at the insertion portion or provided at the insertion portion;
a retained member including a first engaging portion and a second engaging portion which are engaged on a body wall, and a connecting portion connecting the first engaging portion and the second engaging portion and adjusting a distance between the first engaging portion and the second engaging portion; and
a retaining forceps having the retained member at a distal portion thereof, being inserted into the channel, engaging one of the first and second engaging portions on one of a anterior wall of the stomach and a posterior wall of the stomach, engaging the other one of the first and second engaging portions on the other one of the anterior wall and the posterior wall, operating the connecting portion to adjust the distance between the first engaging portion and the second engaging portion with the first or second engaging portions being engaged on the anterior wall or the posterior wall, and retaining the retained member with the first or second engaging portions being engaged on the anterior wall or the posterior wall and the distance between the first engaging portion and the second engaging portion being adjusted.

24. The endoscope apparatus according to claim 23, wherein:
the first and second engaging portions include, respectively, first and second clip portions having first and second grasping elements which are relatively opened or closed, a first prong element provided on an inner side surface of a distal portion of the first grasping element, and a second prong element provided on an inner side surface of a distal portion of the second grasping element;
the connecting portion includes a first thread element connected to the first clip portion, a second thread element connected to the second clip portion, and a binding element binding the first thread element and the second thread element; and
the retaining forceps includes a first clip applicator portion opening or closing, moving and retaining the first clip portion, a second clip applicator portion opening or closing, moving and retaining the second clip portion, and a binding forceps portion moving the binding element relative to the first thread element and the second thread element to adjust a distance between the first clip portion and the second clip portion and retaining the connecting portion with the distance between the first clip portion and the second clip portion being adjusted.

25. The endoscope apparatus according to claim 23, wherein:
the first and second engaging portions include, respectively, first and second rod-shaped portions;
the connecting portion includes a first thread element connected to the first rod-shaped portion, a second thread element connected to the second rod-shaped portion, and a binding element binding the first thread element and the second thread element; and
the retaining forceps includes a hollow needle-shaped forceps element containing on a distal side thereof the first and second rod-shaped portions and containing on a proximal side thereof a pushing element pushing out the first and second rod-shaped portions, and a binding forceps portion moving the binding element relative to the first thread element and the second thread element to adjust a distance between the first rod-shaped portion and the second rod-shaped portion and retaining the connecting portion with the distance between the first rod-shaped portion and the second rod-shaped portion being adjusted.

26. An endoscope apparatus comprising:
an insertion portion perorally;
an observing optical system provided at the insertion portion for observation of a body cavity;
a tube-like member provided integrally or separately at a distal portion of the insertion portion;
a first channel and a second channel which are formed at the insertion portion or provided at the insertion portion so as to open to an inner side of the tube-like member;
at least one first communication portion formed to an outer peripheral wall of the tube-like member to make the interior of the tube-like member communicate with an exterior thereof, through which an anterior wall of stomach is pulled from the exterior of the tube-like member into the interior thereof by suction through one of the first channel and the second channel;
at least one second communication portion formed on the outer peripheral wall of the tube-like member to make the interior of the tube-like member communicate with the exterior thereof, through which a posterior wall of stomach is pulled from the exterior of the tube-like member into the interior thereof by suction through one of the first channel and the second channel,
a retained member having a first engaging portion and a second engaging portion which are engaged on a body wall, and a connecting portion connecting the first engaging portion and the second engaging portion; and
a retaining forceps having the retained member at a distal portion thereof, being inserted into the other one of the first channel and the second channel, engaging one of the first and second engaging portions on one of the anterior wall and the posterior wall pulled into the interior of the tube-like member, engaging the other of the first and second engaging portions on the other of the anterior wall and the posterior wall pulled into the interior of the tube-like member, and retaining the retained member with the first or second engaging portions being engaged on the anterior wall or the posterior wall.

27. The endoscope apparatus according to claim 26, wherein:
the first and second engaging portions include, respectively, first and second rod-shaped portions;
the connecting portion includes a thread-shaped portion; and
the retaining forceps includes a hollow needle-shaped forceps containing on a distal side thereof the retained member and containing on a proximal side thereof a pushing element pushing out the first and second rod-shaped portions.

28. An endoscope apparatus configured to be perorally inserted into a stomach to make a part of a peritoneal cavity side of a fundus of stomach approach a part of the peritoneal cavity side of a false esophagus formed in the stomach, by pulling an anterior wall of stomach and a posterior wall of stomach to each other and joining a part of an inner surface of the anterior wall of stomach and a part of an inner surface of the posterior wall of stomach to each other so as to extend an esophagus.

29. An endoscope apparatus comprising:
an insertion portion perorally inserted into a stomach;
a bending portion provided at the insertion portion and operated to bend; and
an engaging portion provided on the distal portion of the insertion portion and engaged on an inner surface of the fundus of stomach.

30. An endoscope apparatus configured to be perorally inserted into a stomach to fix a part of a peritoneal cavity side of a false esophagus formed in the stomach by pulling an anterior wall of stomach and a posterior wall of stomach to each other and joining a part of an inner surface of the anterior wall of stomach and a part of an inner surface of the posterior wall of stomach to each other so as to extend an esophagus and a part of a peritoneal cavity side of a fundus of stomach made to approach the part of the peritoneal cavity side of the false esophagus.

31. An endoscope apparatus comprising:
an insertion portion perorally inserted into a stomach;
an observing optical system provided at the insertion portion for observation of a body cavity;
a tube-like member provided integrally or separately on a distal portion of the insertion portion;
a first channel and a second channel which are formed at the insertion portion or provided at the insertion portion so as to open to an inner side of the tube-like member;
a communication portion formed to an outer peripheral wall of the tube-like member to make an interior of the tube-like member communicate with an exterior thereof;
a pulling forceps being inserted into one of the first channel and the second channel, and pulling a part of a body wall of the fundus of stomach and a part of a body wall of the false esophagus into the interior of the tube-like member through the communication portion with the part of the body wall of the fundus of stomach and the part of the body wall of the false esophagus being mutually stacked;
a retained member capable of fixing the part of the body wall of the fundus of stomach and the part of the body wall of the false esophagus with the part of the body wall of the fundus of stomach and the part of the body wall of the false esophagus being mutually stacked; and
a retaining forceps having the retained member at a distal portion thereof and being inserted into the other one of the first channel and the second channel, to fix the part of the body wall of the fundus of stomach and the part of the body wall of the false esophagus pulled into the tube-like member, inside the tube-like member, by retaining the retained member.

32. The endoscope apparatus according to claim 31, wherein the pulling forceps includes a grasping forceps having on a distal portion thereof, first and second grasping portions which are relatively opened and closed and which grasps the part of the body wall of the fundus of stomach and the part of the body wall of the false esophagus with they being stacked.

33. The endoscope apparatus according to claim 31, wherein:
the retained member includes first and second rod-shaped portions engaged on the body wall and a thread-shaped portion connecting the first engaging portion and the second engaging portion; and
the retaining forceps includes a hollow needle-shaped forceps containing on a distal side thereof the retained member and containing on a proximal side thereof a pushing element pushing out the first and second rod-shaped portions.

34. An endoscope apparatus perorally inserted into a stomach to wind a fundus of stomach around a peritoneal cavity side of a false esophagus formed in the stomach by pulling an anterior wall of stomach and a posterior wall of stomach to each other and joining a part of an inner surface of the anterior wall of stomach and a part of an inner surface of the posterior wall of stomach to each other so as to extend an esophagus.

35. An endoscope apparatus comprising:
an insertion portion perorally inserted into a stomach;
a bending portion provided at the insertion portion and operated to bend;
an engaging portion provided on a distal portion of the insertion portion and engaged on an inner surface of the fundus of stomach; and
an operating portion provided on the proximal portion of the endoscope to rotate the endoscope about a central axis of the endoscope.

36. An endoscope apparatus comprising:
a tube-like member perorally inserted into a stomach;
a first bending portion provided on the tube-like member and operated to bend; and
an insertion portion inserted into the tube-like member so as to freely advance and retreat and allowed to project from a distal portion of the tube-like member;
a second bending portion provided on the insertion portion and operated to bend.

37. An endoscope apparatus comprising:
means for perorally forming a false esophagus in the stomach by pulling an anterior wall of stomach and a posterior wall of stomach to each other and joining a part of an inner surface of the anterior wall of stomach and a part of an inner surface of the posterior wall of stomach to each other so as to extend an esophagus;
means for perorally making a part of a peritoneal cavity side of a fundus of stomach approach a part of a peritoneal cavity side of a false esophagus; and
means for perorally fixing the part of the fundus of stomach and the part of the false esophagus which are made to approach to each other.

38. An endoscope apparatus comprising:
means for perorally forming a false esophagus in the stomach by pulling an anterior wall of stomach and a posterior wall of stomach to each other and joining a part of an inner surface of the anterior wall of stomach and a part of an inner surface of the posterior wall of stomach to each other so as to extend an esophagus;
means for perorally winding a fundus of stomach around a peritoneal cavity side of the false esophagus; and
means for perorally fixing the part of the wound fundus of stomach and the part of the false esophagus to each other.

39. An endoscope apparatus comprising:
an insertion portion perorally inserted into a stomach;
an observing optical system provided at the insertion portion for observation of a body cavity;
a tube-like member provided integrally or separately at a distal portion of the insertion portion;
a first channel and a second channel which are formed at the insertion portion or provided at the insertion portion so as to open to an inner side of the tube-like member;
a communication portion formed on an outer peripheral wall of the tube-like member to make the interior of the tube-like member communicate with an exterior thereof;
a first retained member having first and second engaging portions engaged with a body wall;
a retaining forceps having the first retained member at a distal portion thereof, being inserted into the first channel, projecting the first retained member from the interior of the tube-like member to the exterior thereof through the communication portion, engaging one of the first and second engaging portions on one of a anterior wall of stomach and a posterior wall of stomach, engaging the other of the first and second engaging portions on the other of the anterior wall and the posterior wall, and retaining the first retained member with the first or second engaging portions being engaged on the anterior wall or the posterior wall;
a bending portion provided at the insertion portion and operated to bend;
an engaging portion provided on the tube-like member and engaged on an inner surface of a fundus of stomach;
a pulling forceps being inserted into the first channel, and pulling a part of a body wall of the fundus of stomach and a part of a body wall of a false esophagus into the interior of the tube-like member through the communication portion with the part of the body wall of the fundus of stomach and the part of the body wall of the false esophagus being mutually stacked;
a second retained member capable of fixing the part of the body wall of the fundus of stomach and the part of the body wall of the false esophagus with the part of the body wall of the fundus of stomach and the part of the body wall of the false esophagus being mutually stacked; and
a retaining forceps having the second retained member at a distal portion thereof and being inserted into the second channel, to fix the part of the body wall of the fundus of stomach and the part of the body wall of the false esophagus pulled into the tube-like member, inside the tube-like member, by retaining the second retained member.

40. The endoscope apparatus according to claim 39, wherein the first channel is formed inside the insertion portion and includes an opening at the distal portion of the insertion portion.

41. The endoscope apparatus according to claim 39, further comprising an elevator formed at the opening portion of the first channel to adjust a direction of advance of the retaining forceps.

42. The endoscope apparatus according to claim 39, wherein the second channel is connected to a suction unit for sucking an interior of the tube-like member.
